(19) United States
(12) Patent Application Publication
(10) Pub. No.: US 2025/0027092 A1
(43) Pub. Date: Jan. 23, 2025

(54) PRODUCTION OF D-LYSERGIC ACID

(71) Applicants: National University of Singapore, Singapore (SG); Imperial College Innovations Limited, London (GB)

(72) Inventors: Wei Jie Garrett WONG, Singapore (SG); Li Rong LIM, Singapore (SG); Maybelle Darlene Kho GO, Singapore (SG); Wen Shan YEW, Singapore (SG); Paul S. FREEMONT, South Kensington, London (GB); David John BELL, London (GB)

(21) Appl. No.: 18/713,604

(22) PCT Filed: Nov. 18, 2022
(86) PCT No.: PCT/SG2022/050840

§ 371 Date: May 24, 2024

(30) Foreign Application Priority Data
Nov. 26, 2021 (SG) 10202113177P

(51) Int. Cl. C12N 15/52; C12N 9/02; C12N 9/04; C12N 9/08; C12N 9/10; C12P 17/18

(52) U.S. Cl. CPC C12N 15/52; C12N 9/0004; C12N 9/0006; C12N 9/0065; C12N 9/0071; C12N 9/1007; C12N 9/1085; C12P 17/182; C12Y 101/01332; C12Y 114/14001; C12Y 201/01261; C12Y 205/01034

(57) ABSTRACT

Modified cells suitable for the production of ergot alkaloids include engineered recombinant cells having one or more genes that code for one or more enzymes in the biosynthetic pathway from tryptophan to D-lysergic acid (DLA). Methods of culturing the engineered recombinant cells can be used for the production of DLA and other ergot alkaloids.

### FIELD OF THE INVENTION

The present invention relates to modified cells suitable for use in the production of ergot alkaloids. More particularly, the present invention provides engineered recombinant cells comprising one or more genes that code for one or more enzymes in the biosynthetic pathway from tryptophan to D-lysergic acid (DLA). The present invention also provides methods of culturing said engineered recombinant cells for the production of DLA and other ergot alkaloids.

### BACKGROUND OF THE INVENTION

The ergot alkaloids are a class of natural products which have been used extensively as therapeutics throughout history. In modern medicine, these compounds and their semi-synthetic derivatives are used particularly in the treatment of several neurological ailments such as Parkinsonism, dementia and hypertension (Lieberman, A., et al., N. Engl. J. Med. 295, 1400-1404 (1976); Winblad, B., et al., Clin. drug Investig. 28, 533-552 (2008); Tandowsky, R., M., Circulation 9, 48-56 (1954)). The ergot alkaloids are broadly classified into three groups—the clavines, ergoamides, and the ergopeptines, all of which are distinguished by the different modifications appended to the core ergoline structure. These compounds are produced by several filamentous fungi from the Ascomycota phylum, but most notably from the parasitic fungus—Claviceps purpurea, more commonly known as the ergot fungus and hence their name (de Groot, A. N., et al., Drugs 56, 523-535 (1998)).

The pharmacological effects of ergot alkaloids have been attributed to the molecular similarity between the ergoline skeleton and the monoamine neurotransmitters, such as adrenaline, dopamine, and serotonin (Pertz, H. & Eich, E., Amsterdam: Harwood Academic Publishers, 411-440 (1999); Mantegani, S., et al., II Farm. 54, 288-296 (1999)). The ergoline pharmacophore is therefore an important scaffold for potential therapeutic discovery and development, particularly in the treatment of neurological and psychiatric disorders. As an illustration, D-lysergic acid diethylamide (LSD), a chemically derived ergot alkaloid, is one of the most potent agonists for the 5-HT_{2A} serotonergic receptor (K_{d}=0.33 nM) (Wacker, D., et al., Cell 168, 377-389, e312 (2017)). The key active pharmaceutical ingredient (API) of these ergoline-derivatives comes from D-lysergic acid (DLA).

Production of therapeutically relevant ergoline-derived pharmaceuticals usually involves the hydrolysis of harvested ergopeptines into DLA, before further semi-synthetic derivatization. To meet the global demand for DLA, 8 tons of ergopeptines and up to 10-15 tons of DLA are produced each year. Approximately 60% of these are produced by the submerged fermentation of specially developed strains of Claviceps purpurea, while the rest are obtained from field cultivation (Cvak, L., 373 (CRC Press, 1999)). However, the key limitations to these existing production methods are: firstly the large variation of ergot alkaloids produced which complicates the downstream extraction workflows and drives up the cost of production (Chen, J.-J., et al., RSC Adv. 7, 27384-27396 (2017)); and second the tendency for these strains to degenerate over the cultivation and preservation processes (Cvak, L., 373 (CRC Press, 1999)).

A number of chemical total synthesis routes towards DLA have also been reported (Liu, H. & Jia, Y., Nat. Prod. Rep. 34, 411-432 (2017)). However, such routes are highly complex, requiring 8 to 19 chemical transformation steps and the implementation of harsh reaction conditions. In addition, the yields are low and the product is often not enantiomerically pure. For example, the highest yielding process requires 19 steps and produces a reported yield of 12% (Umezaki, S., et al., Org. Lett. 15, 4230-4233 (2013)). In contrast, the simplest method is an 8-step process that produces a reported yield of 10.6% but is not enantioselective (Hendrickson, J. B. & Wang, J., Org. Lett. 6, 3-5 (2004)). These issues severely impede the use of chemical synthesis to meet the commercial demand for DLA, which is evident in their lack of use in industry. As seen, the synthesis of these compounds via chemical and biological routes, while of strong industrial relevance, still suffer from several challenges.

Accordingly, there is a significant need to provide improved microbial production systems and methods for producing DLA that overcome, or at least ameliorate, one or more of the disadvantages described above.

### SUMMARY OF THE INVENTION

The present invention provides an isolated recombinant cell comprising one or more genes, wherein each gene codes for an enzyme from the biosynthetic pathway from tryptophan to DLA.

For example, the one or more genes is/are selected from the group consisting of dmaW, easF, easC, easE, easD, easAᵢₛₒₘₑᵣₐₛₑ, and cloA. It will be appreciated that the isolated recombinant cell may comprise one or more orthologue of each gene from the group.

The isolated recombinant cell may comprise at least one dmaW, at least one easF, at least one easE, at least one easD, at least one easAᵢₛₒₘₑᵣₐₛₑ, and at least one cloA gene.

Alternatively, the isolated recombinant cell may comprise at least one dmaW, at least one easF, at least one easE, at least one easC, at least one easD, at least one easAᵢₛₒₘₑᵣₐₛₑ, at least one easG and at least one cloA gene.

The present invention includes a method of culturing the recombinant cell as described herein, in an appropriate culture medium. It will appreciated that the method is for producing 4-Dimethylallyl-L-tryptophan (DMAT), 4-Dimethylallyl-L-abrine (4DMA), Chanoclavine-I, Chanoclavine-I-aldehyde, Agroclavine and/or DLA.

In particular, the method is for preparing DLA.

Advantageously, the engineered recombinant cells of the present disclosure is capable of directly producing DLA and is stable in high density submerged culture. More advantageously, the engineered recombinant cells of the present disclosure eliminates the need to hydrolyze ergopeptines and minimizes the purification and downstream processing complexity by providing the means for direct production of DLA (which is the key ergoline-derivative API). These and other advantages of the present disclosure will become readily apparent to those skilled in this art from the following detailed description.

### BRIEF DESCRIPTION OF THE FIGURES

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 shows a schematic of the complete biosynthetic pathway to the ergopeptides. Beginning with tryptophan through the key intermediate, DLA. Alternative branches of the ergot pathway are indicated by the faded regions. Coloured arrows indicate the stages of the pathway. Green: early pathway common to all ergot producing species, leading to the first major branch point of the pathway at chanoclavine-I-aldehyde. The early pathway consists of four steps, requiring five enzymes (DmaW, EasF, EasC, EasE, and EasD). Blue: middle and late pathway accounting for the diversification of ergoline products found in the various lineages of ergot producing species. This stage of the pathway consists of two steps, requiring at least three enzymes (EasA, EasG, and CloA). Orange: Alternative branches of the pathway that lead to other ergoline derivatives.

FIGS. 2A-2C show the results of the screening of EasE orthologues with a yeast screening strain (YMC17) containing the genes dmaW, easF, and easC stably integrated onto the yeast genome. Introduction of an episomal plasmid expressing the various EasE orthologues identified, to the strain allows for an easy screening platform. FIG. 2A Biosynthetic reaction producing chanoclavine-I from 4DMA. FIG. 2B LC-MS/MS extracted ion chromatograms of the screened EasE orthologues for the ion transition of 257→226 m/z, indicating the production of chanoclavine-I. Region highlighted for the peaks eluting at 121 s, indicating the production of chanoclavine-I. Chromatograms are ordered (bottom to top): GFP control, easE_Ec, easE_Aj, easE_Al, easE_Cf, easE_Pi, easE_Nl, easE_Ei, easE_Ee, easE_Ef. FIG. 2C Relative amounts of chanoclavine-I produced by EasE_Aj and EasE_Ec estimated by the peak area from the MS/MS transition of 257 to 226 m/z. Data are presented as mean values+/−standard deviation. Error bars represent standard deviations calculated from three biological replicates.

FIGS. 3A-3B show the results of screening of EasA orthologues. FIG. 3A Reactions forming the ergoline D ring from chanoclavine-I. Different combinations of enzymes and isoforms (EasA, EasG, and EasH) control the divergence of the tetracyclic ergoclavine products formed at this branchpoint. Orthologues of EasA were screened with episomal plasmids in a strain containing genes for the early pathway enzymes dmaW, easF, easC, and easE integrated onto the yeast genome and regulated by the P_{TEF2}, P_{GPM1}, P_{GAL10}, and P_{GAL1} promoters respectively. FIG. 3B LC-MS chromatograms of the screened EasA orthologues. All selected orthologues were found to produce a compound that co-elutes with the commercially obtained agroclavine standard. Chromatograms appear in the order (bottom to top): YOCE, pCKU-RFP, easA_Pi, easA_Ec, easA_Cpur, easA_Nl, Agroclavine standard.

FIGS. 4A-4D show the results of screening of cloA orthologues. FIG. 4A The putative oxidation and isomerization reactions catalyzed by cloA. Cartoon illustration of the assay design for the screening of cloA product profiles. Strains expressing cloA were fed with commercially obtained agroclavine and analysed for the production of DLA. FIG. 4B LC-MS/MS chromatograms of the products showing the ion transition of 269→223 m/z, produced by the cloA orthologues. Chromatograms appear in the order (bottom to top): pYES2-CT, DLA standard, C. glo (X90), M. rob (392), E. coe (253), C. fus, C. pur (20.1), B. cin, M. acr (SJ7), C. glo (ET3), P. ipo, M. acr (AT5), C. pas, C. pur, N. lol, E. coe (XN6). FIG. 4C Relative amounts of agroclavine consumed by the screened cloA orthologues. FIG. 4D Relative amounts of DLA produced by the various cloA orthologues. Data are presented as mean values+/−standard deviation. Error bars represent standard deviations calculated from three biological replicates.

FIGS. 5A-5D depict the assembling of the functional parts into a DLA-producing yeast strain. FIG. 5A Stepwise extension approach towards pathway construction in the engineered strains, enabling the use of each prior strain as a control for subsequent strains. FIG. 5B LC-MS/MS extracted ion chromatogram monitoring for the ion transition of [M+H]⁺=257→226 m/z, showing the production of chanoclavine-I in both AgcM1B and AgcM2B. FIG. 5C LC-MS/MS extracted ion chromatogram for the ion transition of [M+H]⁺=239→208 m/z, showing the production of agroclavine from AgcM33B. FIG. 5D LC-MS/MS extracted ion chromatogram showing the detected ion transition of [M+H]⁺=269→223 m/z, showing the production of DLA from DLAM33B. Peaks eluted at the time segments (highlighted) correspond to the targeted compounds.

FIG. 6 shows the production of DLA from DLAM33B in 1 and 4 L scale fermentation. The fermentation process was modelled after the induction protocol used in shake flask experiments, with additional galactose and 10×SC-URA media supplemented at defined feeding phases (I, II, III) in a fed-batch mode. 50 mM Ammonium-succinate was supplemented into the culture media from the onset to maintain a pH of 5.8. Both 1 and 4 L culture fermentations achieved a maximum wet cell mass of around 26 g L⁻¹ and a maximum DLA titre of 2.0 mg L⁻¹ and 1.6 mg L⁻¹. Feeding phase I: initial induction phase mimicking the addition of galactose for induction in shake flask experiments. 10× feed solutions were added to the vessel to a final concentration of 1×, at a rate of 3.5 ml min⁻¹. Feeding phase II: sustained feeding phase, a second round of 10× feed solutions were supplemented to the culture to a final concentration of 1× over a period of 52 h. Feeding phase III: starvation phase, no additional carbon or nitrogen source was supplemented. Data are presented as mean values+/−standard deviation. Error bars were calculated from three biological replicates.

FIG. 7 depicts the modifications made to the YeastFab assembly system. Two sets of plasmids were created to improve on the overall workflow: the first was a series of pathway acceptor vectors (level 2) that enables for the screening of pathway modules created to be screened for in E. coli and quickly tested as episomal plasmids in yeast. The second was a series of yeast genome integration vectors to expand the repertoire of integration sites available. The URA3 marker on the integration fragments are designed to be flanked by the homologous sequence URR1, this allows for the removal of the marker by homologous recombination upon the counter-selection with 5-fluoroorotic acid.

FIGS. 8A-8D show the results of the promoter strength measurements at 12 and 18 hours of growth, arranged in descending order by their strengths at 12 hours. FIG. 8A Schematic representation of the promoter reporter plasmid, pGLO3. A promoter released from HCKan_P displaces the RFP cassette in a Golden Gate reaction with Esp3l. When transformed into yeast, the promoter drives the expression of mKOK (orange fluorescent protein). The PCYC1-yeGFP-TCYC1 cassette serves to differentiate cells that harbour the plasmid from those that do not. FIG. 8B Weak promoters-relative strengths to PMDN1 of 1 and below, at 12 hours of growth. FIG. 8C Medium strength promoters—relative strengths between 1 to 6. FIG. 8D Strong promoters—defined by having ≥6 times the strength of P_{MDN1}.

FIGS. 9A-9D depict testing the pathway acceptor plasmids and validating promoter strength data by the production of DMAT. FIG. 9A Illustration of the assembly process to generate pMKU-dmaW series of plasmids. FIG. 9B The first reaction of the ergot alkaloid pathway, catalysed by dmaW producing DMAT. FIG. 9C Overlaid LC-MS chromatograms for 273 m/z of the analysed samples showing peaks of varying sizes corresponding to the amount of DMAT produced. Chromatograms appear in the order (bottom to top): PDC1, PGI1, PYK1, SSA1, TDH2, RPL8, RPL15, PGK1, ENO2, PDA1, GPM1, TPI1, TEF2 and TDH3. FIG. 9D Comparison of the relative amounts of DMAT produced, estimated from the peak area response of the extracted ion chromatograms. Error bars calculated from three biological replicates.

FIGS. 10A-10B show the results of validating the production of DMAT produced from the pMKU-dmaW series of plasmids. Comparison of the retention time and mass spectra of DMAT produced in vivo (FIG. 10A) and in vitro (FIG. 10B) from purified enzymes fed with DMAPP and tryptophan. Proposed structures of the simplest fragment ions observed.

FIGS. 11A-11D depict the results of testing the genome integration vectors via the production of 4DMA. FIG. 11A Cartoon representation of the strain (YMWF) created, containing the expression cassettes: P_{TEF2}-dmaW-T_{ENT2} and P_{GPM1}-easF-T_{PRX1} integrated onto the YMRWΔ15 transposon site. FIG. 11B Reactions catalysed by dmaW and easF to produce 4DMA from tryptophan and DMAPP. Comparison of the retention time and mass spectra of 4DMA produced in vivo (FIG. 11C) and in vitro (FIG. 11D) from purified enzymes supplemented with DMAPP, tryptophan, and S-adenosyl methionine (SAM). Proposed structures of the simplest fragment ions observed.

FIGS. 12A-12C depict the Sequence Similarity Networks (SSN) generated from known gene targets of the ergot pathway using the EFI-EST webtool (Gerlt, J. A., et al., Biochimica Et Biophysica Acta (BBA)—Proteins and Proteomics 1854, 1019-1037 (2015); Zallot, R., et al., Biochemistry 58, 4169-4182 (2019)). In the representation of an SSN, each sequence surveyed is denoted as a node and is linked to other relevant nodes by edges. Each edge represents a predefined degree of similarity. By defining an appropriate alignment score as an edge, nodes can be delineated into isofunctional clusters that group related sequences together in a similar way a multiple sequence alignment draws a consensus sequence. Enzyme sequences within isofunctional clusters could then be expected to be capable of catalyzing similar reactions. Uncharacterized enzymes that are closely related to a known target, could then be identified and tested for desired qualities, which in this work is the functional expression in yeast. Expanding on the hypothetical isofunctional clusters of easE, easA, and cloA allows for the further delineation of the more closely related sequences for the better prediction of their specific activity. FIG. 12A Expanded SSN of the easE isofunctional cluster. Nodes are colour coded by the genus from which the sequences come from; purple: Epichloe, orange: Claviceps, green: Aspergillus, and blue: mainly Penicillium, Pseudogymnoascus, and Trichophyton. FIG. 12B Expanded SSN of the easA isofunctional cluster. Reductase variants of easA showing more sequence divergence and fractioning away from the main cluster. Node colour denote genus of the source organisms; purple: Epichloe, orange: Claviceps, green: Aspergillus, red: Penicillium, brown: Claviceps gigantea and africana, blue: others. FIG. 12C Expanded SSN of the cluster containing the known ergoline-C17 oxidases. Sub-clusters are grouped by the known product profiles of closely related source organisms.

FIGS. 13A-13B show the results of MS/MS fragmentation spectra of easE_Aj and easE_Ec. FIG. 13A MS/MS fragmentation spectra of the peaks eluted at 120.6 seconds from the screen of easE orthologues demonstrating the same product being eluted from both easE_Aj and easE_Ec. FIG. 13B Proposed structures for the two simplest fragment ions, 226 and 208 m/z.

FIG. 14 depict the MS/MS fragmentation spectra of the peaks eluted at 149.6 seconds from the screen of easA orthologues. Comparison of the fragmentation patterns against the commercial agroclavine standard demonstrates the production of agroclavine in strains with the easA_Ec, easA_Nl, and easA_Cp orthologues (at the collision energy of 10 eV).

FIGS. 15A-15B depict the quantification of agroclavine produced from the easA screening strains. FIG. 15A Standard curve of agroclavine established in the negative control (YOCE). The curve was generated by plotting the peak area response for the ion transition of 239→208 m/z against spiked agroclavine concentrations. FIG. 15B Agroclavine titre from the screening strains calculated from the standard curve, measured by the peak area response for the ion transition of 239→208 m/z. Error bars were calculated from three biological replicates.

FIG. 16 depicts the MS/MS fragmentation spectra of the peaks eluted at 126.3 seconds from the screen of cloA orthologs. Comparison of the fragmentation patterns against the commercial DLA standard against the compounds produced from the various cloA orthologs show the production of similar fragmentation ions (at the collision energy of 10 eV).

FIGS. 17A-17D depict the assessment of the performance of selected cloA orthologues in the context of an agroclavine producing yeast chassis. FIG. 17A Cartoon illustration of the experiment performed. FIG. 17B LC-MS/MS chromatograms of the products showing the ion transition of 269→223 m/z. Chromatograms appear in the order (bottom to top): pYES2-CT, cloA_Pi, cloA_Ec, cloA_Cp and DLA. FIG. 17C Standard curve of DLA spiked into samples of the empty vector control. The curve was obtained by plotting the peak area response for the ion transition of 269→223 m/z against spiked DLA concentration. FIG. 17D Quantification of DLA produced from AgcM33B strains expressing the cloA orthologues from an episomal plasmid. Error bars were obtained from three biological replicates.

FIG. 18 shows a schematic representation of the modular introduction of the four segments to sequentially reconstitute the pathway to D-lysergic acid. Following the modified YeastFab workflow, the genetic parts were condensed as transcriptional units on the POT plasmids before assembly as concatenated transcriptional units on the pathway acceptor vectors (p [C/M]K [U/L/H]). Verified pathway segments were then moved into the genome integration vectors (pGAU series). Integrated constructs were verified and cured of the URA3 selection marker for subsequent rounds of integration.

FIG. 19 shows the MS/MS fragmentation spectra of 250 nM agroclavine standard spiked in AgcM2B (top) and peak eluted from AgcM33B (bottom), demonstrating the production of agroclavine in the reconstituted strain. Fragmentation spectrum obtained at the collision energy of 20 eV.

FIG. 20 shows the MS/MS fragmentation spectra of 250 nM DLA standard spiked in AgcM33B (top) and peak eluted from DLAM33B (bottom), demonstrating the production of DLA in the reconstituted strain. Fragmentation spectrum obtained at the collision energy of 20 eV.

FIGS. 21A-21C shows that the ¹³C-tryptophan feedstock for DLAM33B confirms the production of DLA. FIG. 21A Schematic representation of the incorporation of ¹³C-W by DLAM33B to produce ¹³C-DLA. FIG. 21B LC-MS chromatograms showing the [M+H]+ shift in the peak corresponding to the elution of DLA and ¹³C-DLA. FIG. 21C (Top panel) Overlays of the MS/MS spectra obtained from samples supplied with tryptophan (black) and ¹³C-W (red). (Bottom panel) MS/MS difference spectra between samples provided with tryptophan and ¹³C-W, highlighting the +1 m/z shift for the [M+H]+ expected for DLA and its fragmentation ions.

FIGS. 22A-22F depict LC-MS chromatograms showing the incorporation of ¹³C-labelled tryptophan in all intermediates along the ergot alkaloid biosynthesis pathway: FIG. 22A agroclavine (239 m/z) and ¹³C agroclavine (240 m/z); FIG. 22B chanoclavine-I (257 m/z) and ¹³C-chanoclavine-I (258 m/z); FIG. 22C DMAT (273 m/z), 4DMA (287 m/z), ¹³C-DMAT (274 m/z), and ¹³C-4DMA (288 m/z). FIGS. 22D-22F Overlaying the mass spectra between samples fed with ¹³C-tryptophan (Red spectra) and tryptophan (Black spectra) highlight the +1 m/z shift for all precursor ions and their corresponding fragment ions.

FIG. 23 shows the quantification of DLA production titre from the DLAM33B strain by standard addition. The calibration curve was obtained by plotting the area under the curve for the peak response of the MS/MS transition of 269→223 m/z against the final concentration of DLA added to aliquots of the DLAM33B sample. Error bars were calculated from three biological replicates per calibration level.

FIG. 24 depicts a list of promoter sequences characterized in the embodiments of the present invention.

FIG. 25 depicts a list of terminator sequences disclosed in the embodiments of the present invention.

FIG. 26 shows a list of UniProt IDs of ORFs used in the embodiments of the present invention.

FIG. 27 shows a table providing the summary of the exact mass and retention time of chanoclavine-I detected from the screen of easE orthologues in positive mode with an electrospray ionization source (ESI).

FIG. 28 shows a table providing the summary of the exact mass and retention time of agroclavine detected from the screen of easA orthologues in positive mode with an electrospray ionization source (ESI).

FIG. 29 shows a table providing the summary of the exact mass and retention time of DLA detected from the screen of cloA orthologues in positive mode with an electrospray ionization source (ESI).

FIG. 30 shows a table providing the summary of the exact masses measured for the detectable intermediates of the ergot alkaloid pathway with and without the incorporation of ¹³C-2-indole-L-tryptophan, in positive mode with an electrospray ionization source (ESI).

FIG. 31 shows a table providing the summary values of calculated DLA titre from the strain DLAM33BB in the shake flasks.

FIG. 32 shows a list of yeast strains disclosed in the present invention. All strains were derived from S. cerevisiae BY4741 as the base strain.

FIG. 33 shows a table indicating the composition of 10×PBS solution, used for the preparation of 1×PBS (pH 7.4) for screening of cloA orthologs.

FIG. 34 depicts the 1H-NMR assignments for D-lysergic acid in D2O.

FIG. 35 shows a table providing the summary of end-point DLA titres from 4 and 1 L fermentation of DLAM33B.

FIG. 36 depicts a table providing the DNA sequences of the FAD1, PDI1, and the orthologs of dmaW, easF, easC, easE, easD, easA, easG, and cloA genes, as described in the embodiments of the present invention.

FIG. 37 depicts a table providing the corresponding amino acid sequences of the FAD1, PDI1, orthologs of dmaW, easF, easC, easE, easD, easA, easG, and cloA genes as described in the embodiments of the present invention.

### DEFINITIONS

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, “a” or “an” may mean one or more than one unless indicated to the contrary or otherwise evident from the context.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, “enzyme” has its typical meaning in the art and refers to a polypeptide, a protein or in some cases, RNA molecules that acts as a biological catalyst, to bring about a specific biochemical reaction. The term “functional enzyme” therefore refers to an enzyme which retains some or all of its intended activity or function (e.g., biological activity or function, such as enzymatic activity).

The term “functional fragment” refers to a portion of a protein that retains some or all of the activity or function (e.g., biological activity or function, such as enzymatic activity) of the full-length protein, such as, e.g., the ability to bind and/or interact with or modulate another protein or nucleic acid. The functional fragment can be any size, provided that the fragment retains, e.g., the ability to bind and interact with another protein or nucleic acid.

As used herein, “gene product” has its typical meaning in the art and refers to a biochemical material, which is either a protein or RNA molecules, which is produced from the expression of a gene.

As used herein, the term “recombinant cell” means that the cell contains at least one nucleic acid sequence which is not naturally present in the cell or which is naturally present in the cell, but linked to sequences to which it is not naturally linked in the cell such as a promoter to which the nucleic acid sequence encoding a protein is not naturally linked. For example, in the context of the present invention, a recombinant cell differs from the naturally occurring cell in that it contains at least one expression cassette which is not present in the naturally occurring cell.

As used herein, “dmaW” gene refers to a gene sequence that codes for a polypeptide, an enzyme or a gene product which has tryptophan dimethylallyltransferase activity and functions to catalyse the conversion of L-tryptophan to 4-dimethylallyl-L-tryptophan (DMAT).

As used herein, “easF” gene refers to a gene sequence that codes for a polypeptide, an enzyme or a gene product which has N-methyltransferase activity and functions to catalyse DMAT to 4-dimethylallyl-L-abrine (4DMA).

As used herein, “easC” gene refers to a gene sequence that codes for a polypeptide, an enzyme or a gene product which has catalase activity and functions to catalyse 4DMA to chanoclavine-I, in the presence of an EasE activity.

As used herein, “easE” gene refers to a gene sequence that codes for a polypeptide, an enzyme or a gene product which has flavin adenine dinucleotide (FAD)-dependent oxidoreductase activity and functions to catalyse 4DMA to chanoclavine-I, in the presence of an easC activity.

As used herein, “easD” gene refers to a gene sequence that codes for a polypeptide, an enzyme or a gene product which has oxidoreductase activity and functions to catalyse chanoclavine-I to chanoclavine-I-aldehyde.

As used herein, “easA” gene refers to a gene sequence that codes for a polypeptide, an enzyme or a gene product which has either reductase activity (easA_{reductase}) or isomerase activity (easAᵢₛₒₘₑᵣₐₛₑ) or both, and functions to catalyse chanoclavine-I-aldehyde to festuclavine and/or agroclavine, in the presence of an EasG activity.

As used herein, “easA_{reductase}” gene refers to a gene sequence that codes for a polypeptide, an enzyme or a gene product which has reductase activity and functions to catalyse the conversion of chanoclavine-I-aldehyde to festuclavine, in the presence of an easG activity.

As used herein, “easAᵢₛₒₘₑᵣₐₛₑ” gene refers to a gene sequence that codes for a polypeptide, an enzyme or a gene product which has isomerase activity and functions to catalyse the conversion of chanoclavine-I-aldehyde to agroclavine, in the presence of an easG activity.

As used herein, “easG” gene refers to a gene sequence that codes for a polypeptide, an enzyme or a gene product which has oxidoreductase activity and functions to catalyse the conversion of chanoclavine-I-aldehyde to either festuclavine and/or agroclavine, in the presence of easA activity. Particularly, easG catalyses the conversion of chanoclavine-I-aldehyde to festuclavine, in the presence of easA_{reductase} activity and easG catalyses the conversion of chanoclavine-I-aldehyde to agroclavine, in the presence of easAᵢₛₒₘₑᵣₐₛₑ activity.

As used herein, “cloA” gene refers to a gene sequence that codes for a polypeptide, an enzyme or a gene product that functions to catalyse the conversion of agroclavine to oxidised agroclavine products such as, but not limited to, D-lysergic acid (DLA), paspalic acid, lysergol and/or elymoclavine.

As used herein “FAD1” refers to a gene sequence that codes for a polypeptide, an enzyme or a gene product which has FAD synthase activity and functions to catalyse the the adenylation of flavin mononucleotide to form FAD coenzyme.

As used herein, “PDI1” refers to a gene sequence that codes for a polypeptide, an enzyme or a gene product which has protein disulfide isomerase activity.

As used herein, “polypeptide” and “protein” are used interchangeably to denote a polymer of at least two amino acids covalently linked by an amide bond, regardless of length or post-translational modification (e.g., glycosylation or phosphorylation). The term “protein” encompasses a naturally-occurring as well as artificial (e.g., engineered or variant) full-length protein as well as a functional fragment of the protein.

As used herein, the term “orthologue(s)” or “ortholog(s)” are used interchangeably to refer to homologous genes in different species that evolved from a common ancestral gene by speciation. Orthologous genes typically have significant sequence similarity and shared functional domains, inherited from the shared ancestor. The orthologous genes may share at least 25%, 30%, 35%, 40%, 45%, 50%, 55%, 60%, 65%, 70%, 75%, 80%, 85%, 90%, 95%, 96%, 97%, 98% or 99% sequence identity. As those of skill in the art would appreciate, orthologous genes may be identified using bioinformatics approaches such as basic local alignment search tool (BLAST), multiple sequence alignment (MSA), and Enzyme Function Initiative-Enzyme Similarity Tool (EFI-EST) for enzyme prospecting, among others.

### DETAILED DESCRIPTION OF THE INVENTION

A description of exemplary, non-limiting embodiments of the invention follows.

Disclosed herein is an isolated recombinant cell comprising one or more genes, wherein each gene codes for an enzyme from the biosynthetic pathway from tryptophan to D-lysergic acid (DLA). Advantageously, the engineered recombinant cells of the present disclosure serve as effective host strains to directly produce DLA and other ergot alkaloids from central metabolism.

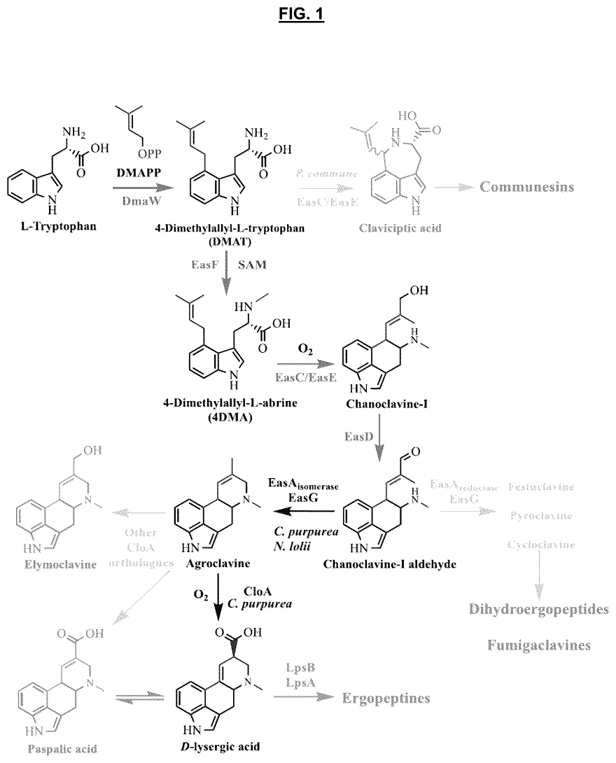

The biosynthetic pathways for ergot alkaloids are shown in FIG. 1. All ergot alkaloids are derived from L-tryptophan and share a common set of early biosynthetic steps that forms the ergoline C-ring. The presence of various isoforms of enzymes from different species of ergot producing organisms, involved in the middle and late biosynthetic steps, determine the product profiles produced by these respective organisms (Cheng, J. Z., et al., Journal of the American Chemical Society 132, 1776-1777 (2010)).

Disclosed herein is the provision of recombinant cells suitable for the production of D-lysergic acid (DLA) and other ergot alkaloids. The recombinant cells of the present disclosure are engineered to comprise at least one gene that codes for an enzyme in the DLA biosynthetic pathway beginning from tryptophan. Also disclosed are methods of culturing said recombinant cells for the production of DLA and other ergot alkaloids.

The present invention provides an isolated recombinant cell comprising one or more genes, wherein each gene codes for an enzyme from the biosynthetic pathway from tryptophan to DLA.

In particular, the one or more genes is/are selected from the group consisting of dmaW, easF, easC, easE, easD, easAᵢₛₒₘₑᵣₐₛₑ, and cloA. It will be appreciated that the isolated recombinant cell may comprise one or more orthologue of each gene from the group.

The isolated recombinant cell may comprise at least one dmaW, at least one easF, at least one easE, at least one easD, at least one easAᵢₛₒₘₑᵣₐₛₑ, and at least one CloA gene.

Examples of easA orthologs include, but not limited to, easA from Claviceps purpurea (easA_Cp), easA from Periglandula ipomoeae (easA_Pi), easA from Neotyphodium lolii (easA_Nl), easA from Epichloe coenophiala (easA_Ec). Examples of EasAᵢₛₒₘₑᵣₐₛₑ orthologs include, but not limited to, EasAᵢₛₒₘₑᵣₐₛₑ from Claviceps purpurea (easA_Cp), EasAᵢₛₒₘₑᵣₐₛₑ from Periglandula ipomoeae (easA_Pi), EasAᵢₛₒₘₑᵣₐₛₑ from Neotyphodium lolii (easA_Nl), EasAᵢₛₒₘₑᵣₐₛₑ from Epichloe coenophiala (easA_Ec).

Examples of cloA orthologs include, but not limited to, cloA from Claviceps paspali (cloA_Cpas), cloA from Neotyphodium lolii (cloA_Nlol), cloA from Periglandula ipomoeae (cloA_Pipo), cloA from Epichloe coenophiala (cloA_XN6, cloA_253), cloA from Claviceps purpurea (cloA_Cpur), cloA from Claviceps fusiformis (cloA_Cfus), cloA from Botrytis cinerea (cloA_Bcin), cloA from Metarhizium acridum (cloA_AT5, cloA_SJ7), cloA from Claviceps purpurea 20.1 (cloA_Cpur) cloA from Metarhizium robertsii (cloA_0X7, cloA_392) and cloA from Colletotrichum gloesporioides (cloA_ET3, cloA_X90).

It will be appreciated that the recombinant cell may comprise one or more orthologue of each gene from the biosynthetic pathway from tryptophan to DLA. For example, the isolated recombinant cell may comprise at least one orthologue of one gene. In particular, the isolated recombinant cell may comprise at least one easE.

Examples of easE orthologs include but are not limited to easE from Epichloe coenophiala (easE_Ec), easE from Aspergillus japonicas (easE_Aj), easE from Aspergillus lentulus (easE_Al), easE from Claviceps fusiformis (easE_Cf), easE from Periglandula ipomoeae (easE_Pi), easE from Neotyphodium lolii (easE_Nl), easE from Epichloe inebrians (easE_Ei), easE from Epichloe elymi (easE_Ee), and easE from Epichloe funkii (easE_Ef) and easE from Aspergillus indologenus.

For example, the easE comprises easE from Epichloe coenophiala (easE_Ec) and/or easE from Aspergillus japonicas (easE_Aj) and/or easE from Aspergillus indologenus. As a further example, the easE may comprise a sequence of at least 80%, at least 85%, at least 90%, at least 95% identity to a sequence selected from the group consisting of SEQ ID NO: 6 to SEQ ID NO: 14.

In particular, the easE from Epichloe coenophiala (easE_Ec) may comprise a sequence at least 80%, at least 85%, at least 90%, at least 95% identity to SEQ ID NO: 14 and/or the easE from Aspergillus japonicas (easE_Aj) or the easE from Aspergillus indologenus comprises a sequence at least 80%, at least 85%, at least 90%, at least 95% identity to SEQ ID NO: 6.

More in particular, the easE comprises easE from Aspergillus japonicas (easE_Aj) or from Aspergillus indologenus. More in particular, the easE comprises a sequence at least 80%, at least 85%, at least 90%, at least 95% identity to SEQ ID NO: 6.

It will be appreciated that the easE in the recombinant cell expresses an enzyme. In particular, the enzyme catalyses conversion of 4-Dimethylallyl-L-abrine (4DMA) to chanoclavine-I.

Further, the isolated recombinant cell comprises at least one easAᵢₛₒₘₑᵣₐₛₑ. For example, the easAᵢₛₒₘₑᵣₐₛₑ comprises easAᵢₛₒₘₑᵣₐₛₑ from Neotyphodium lolii (easA_Nl), easAᵢₛₒₘₑᵣₐₛₑ from Periglandula ipomoeae (easA_Pi), easAᵢₛₒₘₑᵣₐₛₑ from Claviceps purpurea (easA_Cp) and/or easAᵢₛₒₘₑᵣₐₛₑ from Epichloe coenophiala (easA_Ec). As a further example, the easAᵢₛₒₘₑᵣₐₛₑ may comprise a sequence at least 80%, at least 85%, at least 90%, at least 95% identity to a sequence selected from the group consisting of SEQ ID NO: 15 to SEQ ID NO: 18.

In particular, the easAᵢₛₒₘₑᵣₐₛₑ comprises easAᵢₛₒₘₑᵣₐₛₑ from Neotyphodium lolii (easA_Nl), easAᵢₛₒₘₑᵣₐₛₑ from Claviceps purpurea (easA_Cp) and/or easAᵢₛₒₘₑᵣₐₛₑ from Epichloe coenophiala (easA_Ec). In some embodiments, the easAᵢₛₒₘₑᵣₐₛₑ from Neotyphodium lolii (easA_Nl) comprises a sequence at least 80%, at least 85%, at least 90%, at least 95% identity to SEQ ID NO: 15, the easAᵢₛₒₘₑᵣₐₛₑ from Claviceps purpurea (easA_Cp) comprises a sequence at least 80%, at least 85%, at least 90%, at least 95% identity to SEQ ID NO: 18, and the easAᵢₛₒₘₑᵣₐₛₑ from Epichloe coenophiala (easA_Ec) comprises a sequence at least 80%, at least 85%, at least 90%, at least 95% identity to SEQ ID NO: 17.

More in particular, the easAᵢₛₒₘₑᵣₐₛₑ comprises easAᵢₛₒₘₑᵣₐₛₑ from Epichloe coenophiala (easA_Ec). In some embodiments, the easAᵢₛₒₘₑᵣₐₛₑ comprises a sequence at least 80%, at least 85%, at least 90%, at least 95% identity to SEQ ID NO: 17.

It will be appreciated that the easAᵢₛₒₘₑᵣₐₛₑ expresses an enzyme. In particular, the enzyme catalyses conversion of Chanoclavine-I aldehyde to agroclavine.

Further, the isolated recombinant cell comprises cloA. For example, the isolated recombinant cell comprises cloA from Claviceps paspali (CloA_Cpas), cloA from N. lolii (CloA_Nlol), cloA from P. ipomoeae (CloA_Pipo), cloA from E. coenophiala (CloA_XN6) and/or cloA from C. purpurea (CloA_Cpur). In some embodiments, the cloA comprises cloA from E. coenophiala (CloA_XN6) and/or cloA from C. purpurea (CloA_Cpur).

As a further example, the cloA from E. coenophiala (CloA_XN6) may comprise a sequence at least 80%, at least 85%, at least 90%, at least 95% identity to SEQ ID NO: 27 or 28 and the cloA from C. purpurea (CloA_Cpur) comprises a sequence at least 80%, at least 85%, at least 90%, at least 95% identity to SEQ ID NO: 22, 23 or 24.

In particular, the cloA comprises cloA from C. purpurea (CloA_Cpur). More in particular, the cloA from C. purpurea (CloA_Cpur) comprises a sequence at least 80%, at least 85%, at least 90%, at least 95% identity to SEQ ID NO: 23.

It will be appreciated that the cloA expresses an enzyme in the recombinant cell. In particular, the enzyme catalyses conversion of agroclavine to D-lysergic acid.

The recombinant cell may comprise more than one gene encoding for an enzyme from the biosynthetic pathway from tryptophan to D-lysergic acid (DLA).

For example, the recombinant cell may comprise at least one easE, at least one easAᵢₛₒₘₑᵣₐₛₑ and at least one cloA.

For this example, the easE may comprise easE from Aspergillus japonicas (easE_Aj) or easE from Aspergillus indologenus, the easAᵢₛₒₘₑᵣₐₛₑ may comprise easAᵢₛₒₘₑᵣₐₛₑ from Epichloe coenophiala (easA_Ec) and the cloA may comprise cloA from C. purpurea (CloA_Cpur). The recombinant cell may further comprises at least one dmaW, at least one easF, and at least one easD. In particular, the dmaW may comprise a sequence at least 80%, at least 85%, at least 90%, at least 95% identity to SEQ ID NO: 1, the easF may comprise a sequence at least 80%, at least 85%, at least 90%, at least 95% identity to SEQ ID NO: 2; and the easD may comprise a sequence at least 80%, at least 85%, at least 90%, at least 95% identity to SEQ ID NO: 4.

It will be appreciated that the dmaW, easF and easD each expresses a respective enzyme. In particular, the each respective enzyme is functional.

In addition to comprising at least one easE, at least one easAᵢₛₒₘₑᵣₐₛₑ and at least one cloA, the recombinant cell further comprises at least one easC and/or at least one easG. In particular, the easC comprises a sequence at least 80%, at least 85%, at least 90%, at least 95% identity to SEQ ID NO: 3 and easG comprises a sequence at least 80%, at least 85%, at least 90%, at least 95% identity to SEQ ID NO: 5.

As a particular embodiment, there is provided an isolated recombinant cell comprising at least one dmaW, at least one easF, at least one easE or easC, at least one easD, at least one easAᵢₛₒₘₑᵣₐₛₑ or easG, and at least one cloA gene.

As a second particular embodiment, there is provided an isolated recombinant cell comprising at least one dmaW, at least one easF, at least one easE, at least one easD, at least one easAᵢₛₒₘₑᵣₐₛₑ, and at least one cloA gene.

As a third particular embodiment, there is provided an isolated recombinant cell comprising at least one dmaW, at least one easF, at least one easC, at least one easD, at least one easAᵢₛₒₘₑᵣₐₛₑ, at least one easG, and at least one cloA gene.

It will be appreciated that the dmaW comprises a sequence at least 80%, at least 85%, at least 90%, at least 95% identity to SEQ ID NO: 1; easF comprises a sequence at least 80%, at least 85%, at least 90%, at least 95% identity to SEQ ID NO: 2; easE comprises a sequence at least 80%, at least 85%, at least 90%, at least 95% identity to SEQ ID NO: 6; easC comprises a sequence at least 80%, at least 85%, at least 90%, at least 95% identity to SEQ ID NO: 3; easD comprises a sequence at least 80%, at least 85%, at least 90%, at least 95% identity to SEQ ID NO: 4; easAᵢₛₒₘₑᵣₐₛₑ comprises a sequence at least 80%, at least 85%, at least 90%, at least 95% identity to SEQ ID NO: 17; easG comprises a sequence at least 80%, at least 85%, at least 90%, at least 95% identity to SEQ ID NO: 5; and cloA comprises a sequence at least 80%, at least 85%, at least 90%, at least 95% identity to SEQ ID NO: 23.

The recombinant cells of the present disclosure can be further designed to incorporate gene(s) that enhances the production of FAD. In some embodiments, the isolated recombinant cell as described herein further comprises FAD1 and PDI1 genes. In some embodiments, the isolated recombinant cell the isolated recombinant cell further comprises multiple copies of FAD1 and PDI1 genes. In some embodiments, the FAD1 comprises a sequence at least 80%, at least 85%, at least 90%, at least 95% identity to SEQ ID NO: 35 and PDI1 comprises a sequence at least 80%, at least 85%, at least 90%, at least 95% identity to SEQ ID NO: 36.

It will be appreciated that the isolated recombinant cell is capable of expressing the one or more genes. The expression of the one or more genes produces respective gene product(s). In particular, the respective gene product(s) are functional. More in particular, the respective gene product(s) are respective enzyme(s).

It will be appreciated that any suitable cell may be used for the recombinant cell. The recombinant cell may be any eukaryotic recombinant cell. For example, the recombinant cell may be a recombinant yeast cell. In particular, the recombinant cell may be from Saccharomyces sp. More in particular, the recombinant cell may be a recombinant Saccharomyces cerevisiae.

In one particular embodiment, the isolated recombinant yeast cell is a DLAM33B strain.

Provided herein is also a method of culturing the recombinant cell of the present disclosure. In one aspect, there is provided a method of culturing the recombinant cell of any one of the earlier aspects in an appropriate culture medium. In particular, the method comprises incubating the recombinant in an appropriate culture medium.

It would be appreciated by a person of skill in the art that the recombinant cells of the present disclosure may be cultured in medium such as, but not limited to, SC medium, SM medium, YPD medium, YPG medium, and YPAD medium. In some embodiments, the medium is SC medium.

In some embodiments, the method is suitable for producing 4-Dimethylallyl-L-tryptophan (DMAT), 4-Dimethylallyl-L-abrine (4DMA), Chanoclavine-I, Chanoclavine-I-aldehyde, Agroclavine and/or D-lysergic acid.

In some embodiments, the method is suitable for preparing D-lysergic acid.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

### Materials and Methods

### Cultivation, Media and Strain

E. coli XL1Blue (Stratagene), E. coli NEB Stable (New England Biolabs) and S. cerevisiae strain BY4741 were the base strains used in this study. E. coli constructs were grown in lysogeny broth (LB) at 37° C. with the appropriate antibiotics. Competent E. coli cells were prepared and transformed following the Inoue protocol, with the modification that cells were grown at 30° C. to mid-logarithmic phase before further processing (Inoue, H., et al.; Gene 96, 23-28 (1990)). Yeast strains were grown in either Yeast extract-Peptone-Dextrose (YPD) or in Synthetic-Complete (SC) media omitting the appropriate nutrient for selection (Treco, D. A. & Lundblad, V.; Curr. Protoc. Mol. Biol. 23, 13.11.11-13.11.17 (1993)). Transformation of plasmids and DNA fragments for chromosomal integration in S. cerevisiae were performed using the Lithium acetate/PEG-3350/single-stranded carrier DNA protocol (Gietz, R. D. & Schiestl, R. H.; Nat. Protoc. 2, 1 (2007)).

### Assembly of Modified YeastFab Plasmids

The pathway acceptor plasmids and genome integration plasmids were constructed through Gibson Assembly (Gibson, D. G., et al.; Nature methods, 6 (5), 343-345 (2009)). Pathway acceptor plasmids were assembled from four fragments, each fragment holding some core elements (Kan^{R} and ColE1 origin, RFP expression cassette with insert and release sites, a yeast origin of replication, and a yeast selection marker). Genome integration plasmids were assembled similarly from five fragments (URA3 selection marker flanked by URR sites, RFP expression cassette with insert sites, Amp^{R} and ColE1 origin, upstream and downstream genome integration homology regions). All fragments were created through PCR amplification (Takara PrimeSTART) from appropriate sources. The created plasmids were verified by restriction digest and Sanger sequencing.

### Golden-Gate Assembly of Parts and Pathways

The Golden-gate assembly used in this study predominantly is in accordance with existing published protocols, save for a few modifications (Engler, C., et al., PloS one 4, e5553 (2009); Tan, Y. Q., et al., Biomacromolecules (2021)). Reactions were prepared as 10 μL pots, each containing 1 μL 10×T4 ligase buffer (New England Biolabs), 1 μL 100× bovine serum albumin (New England Biolabs), 5 U of restriction enzyme (BsaI-HFv2 or Esp3l, New England Biolabs), 10 U T4 DNA ligase (New England Biolabs), 15 ng of destination plasmid, 1 μL per insert and brought to 10 μL with sterile deionized distilled water (ddH₂O). The assembly reactions were cycled beginning with 37° C. for 5 minutes followed by 25° C. for 10 minutes for 25 cycles, before finishing with 55° C. for 20 minutes and 80° C. for a further 20 minutes. The reaction mixture was subsequently directly used for the transformation of chemically competent XL1Blue (Level 0/1 constructs) or NEB Stable (Level 2 and beyond).

Yeast promoter and terminator parts were PCR amplified from S. cerevisiae S288C genomic DNA and cloned into HcKan_P and HcKan_T respectively. For simplicity, promoter and terminator sequences were defined as the region 500 base pairs (bp) upstream or downstream of the ORF used for naming the genetic element. The genes encoding the enzymes of the pathway were codon optimized for yeast expression, synthesized, and cloned into HcKan_O. The genes for FAD1 and PDI1 were PCR amplified (Takara PrimeSTAR™) from S. cerevisiae S288C genomic DNA and cloned into HcKan_O. All level 0 and level 1 constructs assembled were verified by colony PCR using Taq DNA polymerase (New England Biolabs) and Sanger sequencing, while level 2 constructs and genome integration constructs were verified by colony PCR.

### Small-Scale Yeast Culture for the Production of Ergot Alkaloids

For each assayed construct, three isolated colonies either freshly transformed or streaked were used to inoculate a 10 mL pre-culture in a 50 ml tube of the appropriate growth media and grown for at least 18 hours at 30° C. in a shaking incubator at 210 rpm. These cultures were then back diluted to a final OD₆₀₀ of 0.0125 in 10 ml of the appropriate SC media supplemented with 0.1% (w/v) D-glucose. Cultures were further grown to an OD₆₀₀ of 0.8 before galactose was added to a final concentration of 2% (w/v) from a filter-sterilized 20% (w/v) stock solution. The caps of the culture tubes were then replaced with autoclaved aluminium foil caps and grown for 120 hours at 24° C. Cultures are subsequently pelleted by centrifugation at 4000 rpm for 10 minutes. A 1 mL aliquot of the collected supernatant from each sample was then filtered through a 0.2 um PTFE syringe filter and analyzed using liquid chromatography-tandem mass spectrometry (LC491 MS/MS).

Fed-Batch Fermentative Production of DLA from Engineered Yeast

Culture media used in both 4 and 1 L fermentations consisted primarily of SC-URA with 0.1% (w/v) glucose and 50 mM ammonium-succinate, pH 5.8 (SCUS). Two feed solutions were used, Feed 1 consisted of 10×SC-URA amino acids mix and 10× Yeast nitrogen bases; Feed 2 consisted of 20% (w/v) galactose and 100 mM ammonium-succinate, pH 5.8.

The seed culture was prepared by growing a single colony of DLAM33B from a freshly streaked plate in 10 mL SC-URA media with 2% (w/v) glucose at 30° C. overnight. Fermentation was initiated by inoculating the fermentation vessel (INFORS HT Minifors 2, Bottmingen, Basel, Switzerland) filled with 2 L or 500 mL of SCUS with the seed culture to a final OD₆₀₀ of 0.0125.

The fermentation process begins with an initial outgrowth phase (phase 0) at 30° C. with stirring at 1000 rpm and compressed air (Ekom DK40 2V, Singapore) was used to supply an airflow of 1 vessel volume per minute (either 4 L min⁻¹ or 1 L min⁻¹), Dissolved oxygen (DO) level was maintained at >90% saturation through an automated cascade to increase stirring rate up to 1500 rpm and to increase airflow up to 2 vessel volume per minute (8 L min⁻¹ or 2 L min⁻¹). This phase allows for the depletion of the glucose present and for the culture to propagate to an adequate cell density for induction. After 24 hours, the induction phase (phase I) is initiated by pumping both Feed 1 and 2 into the vessel at 3.5 mL min⁻¹, until the volume of each feed pumped in to the starting fermentation culture volume is 1 part to 8 parts. The temperature was also lowered to 24° C. for the induction of the pathway. Phase 0 and I directly mimic the conditions used for pathway induction at the shake flask scale. Phase II was pre-programmed to begin after 20 hours of phase I, when the galactose supplemented in phase I is expected to begin its exponential decline (Sanchez, R. G., et al., Microbial Cell Factories 9, 1-8 (2010)) In this phase, a steady low-level flow rate of 0.05 mL min⁻¹ (1 L) or 0.18 mL min⁻¹ (4 L) for both Feed 1 and 2 is maintained over 28 hours to maintain sufficient nutrients in the culture, as well as ensure sufficient expression of pathway genes. At phase III or the starvation phase, no additional feed was supplemented, and the fermentation was allowed to carry on for an additional two days. The fermentation was monitored by drawing 10 mL samples daily and assessed for wet cell mass (WCM) and DLA production titre.

Screening of cloA Orthologues

Genes encoding the various cloA orthologues were synthesized and cloned (BioBasic) into the pYES2/CT vector (Invitrogen). Cells were cultured and expression was induced as earlier described with the exception that a 1 mL aliquot of induced cells were removed from each tube and transferred into a 1.5 mL microcentrifuge tube. Cells were then pelleted by centrifugation at 21000 g for 1 minute. The pellet was then resuspended in 1 mL of phosphate buffered saline (PBS), pH 7.4. Agroclavine was then spiked into each tube to a final concentration of 5 μM and incubated at 30° C. overnight. The PBS incubations were then pelleted by centrifugation and the supernatant from each sample was filter sterilized using a 0.2 um PTFE syringe filter and similarly analyzed using liquid chromatography-tandem mass spectrometry (LC-MS/MS).

¹³C-Labelled Experiments with ¹³C-2-Indole-L-Tryptophan

A stock solution of ¹³C-2-indole-L-tryptophan (Sigma) was prepared by dissolving the powder in 20% (w/V) galactose to a final concentration of 10 mM and filter-sterilized. ¹³C-Labelling of DLA and its associated pathway intermediates were carried out using the same protocol described for the production of ergot alkaloids, but were induced with the stock solution of ¹³C-2-indole-L-tryptophan in galactose instead of just galactose alone, to a final concentration of 1 mM ¹³C-2-indole-L-tryptophan and 2% (w/v) galactose. The negative control used was a similar preparation with L-tryptophan (Sigma) in place of ¹³C-2-indole-L-tryptophan.

### Analysis of Ergot Alkaloids by High-Performance Liquid Chromatography-Tandem Mass Spectrometry (HPLC-MS/MS)

All samples were analyzed using the Agilent 1290 Infinity LC system coupled to an Agilent 6550 iFunnel QTOF with an electrospray ionization source. Samples were separated using an Agilent InfinityLab Poroshell 120 EC-C18 column with the dimensions of 2.1×100 mm, 1.9 μm particle size. The mobile phases used consisted of: A, containing water with 0.1% formic acid; and B, containing acetonitrile with 0.1% formic acid. Chromatography was carried out over a constant flow rate of 0.5 mL/min, 1 μL injection volume, with a stepped gradient as follows: 95% A/5% B for 0.6 min, 65% A/35% B to 2.6 min, 1% A/99% B to 4.6 min. The column was washed with 100% B for 2 min before re-equilibrating to 95% A/5% B for 1 min.

Mass data acquisition was set to targeted MS/MS mode using fixed polarity (positive), from the eluent beginning from 1 min into the run and ending at 4.5 min. Instrument parameters were set to run at; source gas temperature and flow of 200° C. and 10 L/min, sheath gas temperature and flow of 350° C. and 10 L/min, nebulizer pressure at 50 psig. Capillary and nozzle voltages were set to 4000 V and 0 V respectively. MS1 was set to a mass range of 40-1000 m/z, at a scan rate of 3 spectra/second. MS2 was set to a mass range of 40-1000 m/z, at a scan rate of 6 spectra/second using a fixed collision energy of either 10 eV for screening experiments or 20 eV for the analysis of the reconstituted strains.

The targeted mass for the screening of easE orthologs to produce chanoclavine-I was set to 257.1648 m/z, on a narrow isolation bandwidth (1.3 amu). In the screen for isomerase variants of easA, the targeted mass was set to 239.1543 m/z, on a narrow isolation width (1.3 amu). For the screening of cloA orthologs, the targeted masses were set for: 1) 239.1543 m/z, narrow isolation width (1.3 amu); 2) 269.1285 m/z, narrow isolation width (1.3 amu). In the assay for the reconstitution of the ergot alkaloid pathway, the targeted masses were set for: 1) 287.1754 m/z, narrow isolation width (1.3 amu); 2) 257.1648 m/z, narrow isolation width (1.3 amu); 3) 239.1543 m/z, narrow isolation width; 4) 269.1285 m/z, narrow isolation width (1.3 amu). To detect the incorporation of ¹³C-labelled-tryptophan into the reconstituted pathway, the targeted masses were set for: 1) 269.1285 m/z, narrow isolation width (1.3 amu), 2) 270.1318 m/z, narrow isolation width. All data analysis and instrument control were performed using the Mass Hunter software suite (Agilent).

Determination of compound identities were performed by the comparison of retention time and MS/MS product ion spectrum against commercially obtainable standards where available (D-lysergic acid; Chiron) (Agroclavine; Chiron/Toronto Research Chemicals). Determination of 4DMAT, 4DMA and chanoclavine-I was performed by the comparison of the retention times and MS/MS product ion spectrum against in vitro biosynthesized products of purified dmaW, easF, easC and cell extracts expressing easE_Aj. Quantification of agroclavine and DLA produced was performed using either a calibration curve established from the commercial standards or by standard addition. Agroclavine was quantified by monitoring the transition of the 239 m/z precursor ion to 208 m/z and DLA was quantified by monitoring the transition of the 269 m/z precursor ion to 223 m/z. Linear regression analysis of the standard curves were performed using Graphpad Prism version 7.00 for Windows (Graphpad Software, San Diego, CA).

### Bioinformatics Analysis

All SSNs used in this study were generated using a protein sequence query for the initial BLAST search (Option A), with the parameters set to retrieve a maximum of 9000 sequences with a minimum alignment E-value of 5, through the EFI-EST webtool (Zallot, R., et al., Current opinion in chemical biology 47, 77-85 (2018)). The initial network was calculated by defining an edge to represent a relationship with an alignment score greater than or equal to the equivalent of 40% sequence identity. Each SSN was subsequently individually refined by increasing the edge score until the “hairballs” fragmented into smaller hypothetical iso-functional clusters. Manipulation and visualization of SSNs were performed using the Cytoscape software (Shannon, P., et al., Genome research 13, 2498-2504 (2003)). Selected sequences were then retrieved from the Uniprot database using the associated Uniprot numbers from the SSN.

### Flow Cytometry Analysis of Yeast Promoter Library

The promoter reporter plasmid pGlo3 containing the promoter library inserts were transformed into yeast BY4741 cells. From the transformants, three individual colonies were picked and cultured in liquid SC-URA for 30 h at 25° C., 220 rpm until saturation (OD≈3). Subsequently, fresh media was inoculated with saturated cell culture in 1:200 dilution and grown for 12 h at 25° C., at which point the optical density at 600 nm (OD₆₀₀) reached approximately 0.9 to 1.2, which corresponds to the exponential growth phase. To 180 μL of ice-cold PBS was added 20 μL of the cell culture and the 96-well plate holding the samples was kept at 4° C. until flow cytometry analysis, which was no longer than 4 h later. The remaining cell cultures were grown for a further 6 h at 25° C. until the OD₆₀₀ reached ˜2.0-2.5, corresponding to early stationary phase. Similarly, 20 μL of the culture was diluted in 180 μL ice-cold PBS and kept cold until analysis.

The yeGFP and mKOκ emissions of each individual cell were measured using the BD Accuri™ flow cytometer. For each sample, 20 000 cells were measured, and the flow rate was adjusted to roughly 2000 cells·s⁻¹. For each batch of samples, a strain expressing P_{PGK1} driven yeGFP and another strain expressing mKOκ on high copy 2μ plasmids were included as fluorescence compensation controls and a strain containing plasmid pCKU (which does not express any fluorescent protein) served to account for background fluorescence. Results were analyzed by using the FlowJo (Version 10) software. Fluorescence bleed-through between the green and orange emission channels were first compensated for using the 2μ plasmid controls. Subsequently, the signal readout from promoter activity was obtained as the geometric means of the orange emissions of the plasmid harboring strains (identified by yeGFP emission) minus the background orange emission as measured by using the pCKU strain.

Expression and Purification of dmaW and easF from E. coli for In Vitro Assays

The genes encoding these two proteins were cloned into pET15B vectors and recombinantly expressed in E. coli BL21 (DE3) cells. Expression was carried out by growing cells in 2YT media to an OD₆₀₀ of 0.7 at 37° C., prior to induction with IPTG (2 mM) at 20° C. for 20 hours. The cells were then pelleted by centrifugation at 4° C., 5000 rpm for 10 mins. Pelleted cells were resuspended in binding buffer (5 mM imidazole, 0.5 M NaCl, 20 mM Tris-HCl, pH 7.9) at 4° C. and lysed via sonication. Cell debris was then clarified by centrifugation at 15000 rpm for 20 mins at 4° C. All subsequent purification steps were done at 4° C. The clarified cell supernatant was then added to 200 μL of Ni²⁺-NTA chelating sepharose resin equilibrated in binding buffer. This was incubated for 30 mins with shaking at 140 rpm. The resin was washed three times with 2 mL wash buffer (60 mM imidazole, 0.5 M NaCl, 20 mM Tris-HCl, pH 7.9) for 5 mins with shaking at 90 rpm for each washing step. The bound protein was eluted twice from the resin with 500 μL of His-Elute buffer (100 mM L-histidine, 0.5M NaCl, 20 mM Tris-HCl, pH 7.9) for 10 mins with shaking at 70 rpm. The fractions were analyzed using SDS-PAGE and those containing the protein(s) were concentrated using 3 kDa molecular weight cut-off (MWCO) Ultra-0.5 spin filters (Amicon). The purified solution was then dialyzed against the storage buffer containing 50 mM Tris-HCl, pH 7.5, 5 mM CaCl₂, 50% glycerol and stored at −20° C.

The in vitro biosynthesis of DMAT was prepared in a 60 μL reaction volume containing 50 mM Tris-HCl, pH 7.5, 5 mM CaCl₂, 1 mM L-tryptophan, 1 mM DMAPP, and 10 μL purified dmaW. The reaction was incubated at 30° C. for 18 hrs. The reaction was stopped by filtering off the enzyme using a 3 kDa MWCO Ultra-0.5 spin filters (Amicon). The sample was either stored at −20° C. or immediately analysed using LC-MS. The in vitro biosynthesis of 4DMA was prepared similarly but with the addition of 1 mM SAM and 10 μL purified easF.

### Nuclear Magnetic Resonance (NMR) Analysis of Biosynthesized DLA

Cell culture to produce DLA for NMR analysis was performed as described earlier. DLA was purified from the culture media (8 L) by first lyophilizing the collected clarified media. The dried culture was subsequently reconstituted in a 300 mL of ddH₂O and purified by liquid chromatography using an AKTA Pure 25M (Cytiva) affixed with a C-18 preparative column (Agilent Zorbax Eclipse XDB-C18, semi-preparative; 9.4×646 250 mm, 5 μm particle size). The mobile phases used consisted of: A, water with 0.1% trifluoroacetic acid; and B, acetonitrile with 0.1% trifluoroacetic acid. Semi-preparative chromatography was carried out over a constant flow rate of 2 mL/minute, 2 mL injections, with a stepped gradient as follows: 90% A/10% B for 10 minutes, 90% A/10% B to 80% A/20% B over 50 minutes. Between runs, the column was washed with 100% B for 4 column volumes (CV) (80 mL) of 100% B at a flow rate of 10 mL/min, before re-equilibrating to 90% A/10% B for 4 CV at a flow rate of 2 mL/min. Elution of DLA was 3 monitored by absorbance at 310 nm, fractions corresponding to peaks at 310 nm were collected and pooled. The pooled fractions were concentrated by lyophilization and 150 μL aliquots were taken for LC-MS/MS analysis of the purity and confirmation of the presence of DLA. The remaining pooled fractions were lyophilized to dryness and stored at −20° C.

The sample for NMR analysis was prepared by adding 2 mL of D₂O (Sigma) to the combined dried fractions, any insoluble material was removed by centrifugation at 4000 rpm, 20 minutes. Subsequently, 1 mL of D₂O saturated with the sample was used for analysis of the 1H-NMR spectra using a Bruker AVANCE 500 MHZ NMR spectrometer at the Department of Chemistry, National University of Singapore.

### Results

### Biosynthetic Resolution of the Ergot Alkaloid Pathway

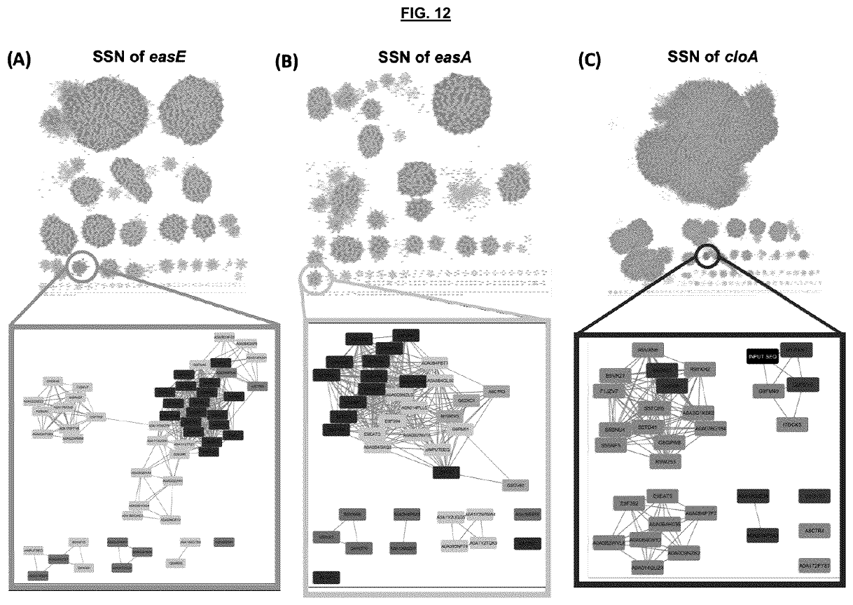

The complete biosynthesis of DLA from L-tryptophan requires eight enzymes encoded by the following genes—DmaW, EasF, EasC, EasE, EasD, EasA, EasG, and CloA. (Chen, J.-J., et al., RSC Advances 7, 27384-27396 (2017)). The transformations from DmaW to EasD have been biochemically characterized (FIG. 1) (Chen, J.-J., et al., RSC Advances 7, 27384-27396 (2017); Metzger, U., et al., Proceedings of the National Academy of Sciences 106, 14309-14314 (2009); Rigbers, O. & Li, S.-M., Journal of Biological Chemistry 283, 26859-26868 (2008); Nielsen, C. A., et al., Microbial cell factories 13, 1 (2014); Wallwey, C., et al., Archives of microbiology 192, 127-134 (2010)). All ergot alkaloids are derived from L-tryptophan and share a common set of early biosynthetic steps that forms the ergoline C-ring. The presence of various isoforms of enzymes from different species of ergot producing organisms, involved in the middle and late biosynthetic steps, determine the product profiles produced by these respective organisms (Cheng, J. Z., et al., Journal of the American Chemical Society 132, 1776-1777 (2010)). Some of the enzymes have, however, been reported to be refractory toward heterologous expression (Nielsen, C. A., et al., Microbial cell factories 13, 1 (2014); Cheng, J. Z., et al., Journal of the American Chemical Society 132, 1776-1777 (2010)). Therefore, alternative orthologues were sought for these enzymes (encoded by genes easE, easA, and cloA) (FIG. 12) to reconstitute the pathway, using the EFI-EST (Gerlt, J. A., et al., Biochimica Et Biophysica Acta (BBA)—Proteins and Proteomics 1854, 1019-1037 (2015); Zallot, R., et al., Biochemistry 58, 4169-4182 (2019)).

### Example 2: Screening for Functional Expression of easE in Yeast

The enzymes EasC and EasE have been reported to both, be essential in the conversion of 4DMA to chanoclavine-I in earlier publications from several groups (Nielsen, C. A., et al., Microbial cell factories 13, 1 (2014); Lorenz, N., et al., Appl. Environ. Microbiol. 76, 1822-1830 (2010); Goetz, K. E., et al., Current genetics 57, 201 (2011); Kozikowski, A. P., et al., Journal of the American Chemical Society 115, 2482-2488 (1993)). However, EasE from most ergot producing fungi have been shown to have non-optimal activity in heterologous yeast systems (Nielsen, C. A., et al., Microbial cell factories 13, 1 (2014)). To date, only the EasE orthologue from Aspergillus japonicus (EasE_Aj), reported by Nielsen, C. A., et. al. (2014), has been functionally expressed in S. cerevisiae.

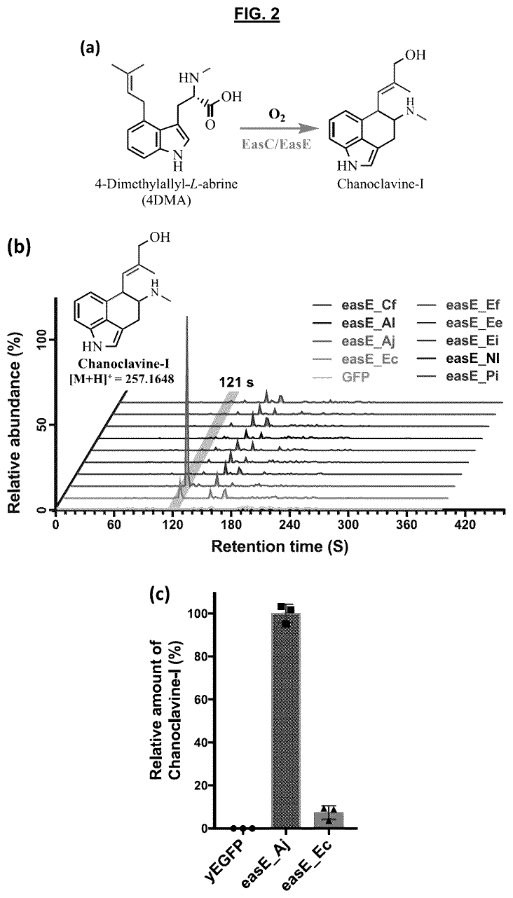

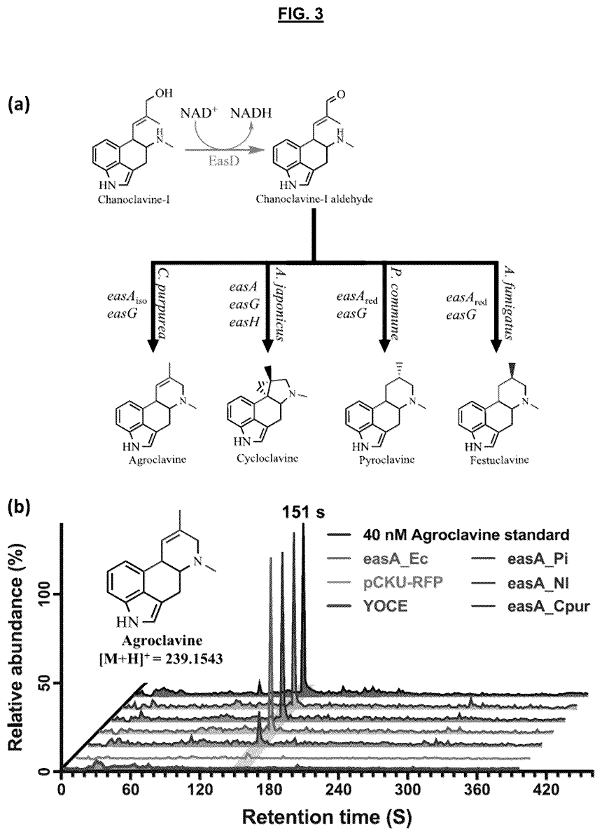

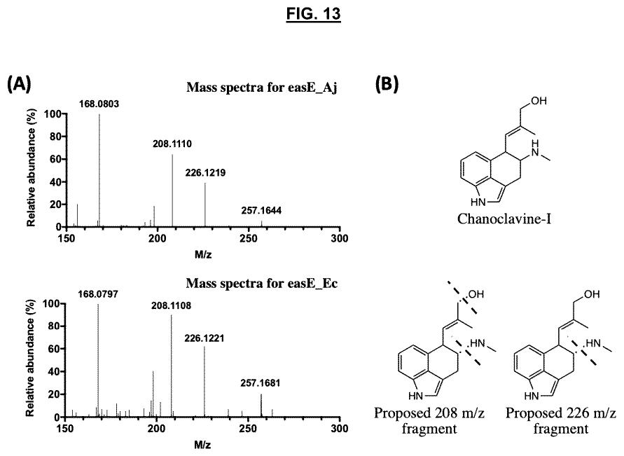

Therefore, to identify additional active EasE orthologues, this orthologue was used to generate a Sequence Similarity Network (SSN). Then, the putative isofunctional cluster around EasE_Aj was identified and eight sequences were selected to screen for expression and enzymatic function. To facilitate this screen, a modified strain (YMC17; Supplementary Table 9) was created with the genes: dmaW, easF, and easC; stably integrated into its genome at the YMRWδ15 site. The eight EasE orthologues were then transformed into YMC17, on an episomal vector. Out of the eight orthologues, only the orthologues from Epichloe coenophiala (EasE_Ec) and EasE_Aj exhibited detectable production of chanoclavine-I (FIG. 2B, FIG. 13). Comparisons of the relative amounts of chanoclavine-I produced by EasE_Ec were 10 times lower compared to that from easE_Aj (FIG. 2C). Nevertheless, both sequences could be used to complete the ergot alkaloid pathway in yeast.

### Example 3: Identifying Isomerase Variants of easA

The next biosynthetic step in the construction of the DLA pathway involves the branch point linking the tricyclic clavines to the tetracyclic ones. The isoforms of EasA, from different lineages of ergot alkaloid producing organisms, catalyze either a reduction or a cis-trans isomerization across the C8-C9 double bond of the ergoline moiety to position the aldehyde group for EasG to then link it with the methylamino group to form the ergoline D ring (Floss, H. G., et al., Journal of the American Chemical Society 90, 6500-6507 (1968)). The reduction or retention of the C8-C9 double bond at the end of this process depends on the EasA isoform, diverging the pathway toward either agroclavine, festuclavine, pyroclavine, or if a particular orthologue of EasH is present, cycloclavine (FIG. 3A) (Cheng, J. Z., et al., Journal of the American Chemical Society 132, 1776-1777 (2010); Floss, H. G., et al., Journal of the American Chemical Society 90, 6500-6507 (1968); Cheng, J. Z., et al., Journal of the American Chemical Society 132, 12835-12837 (2010)).

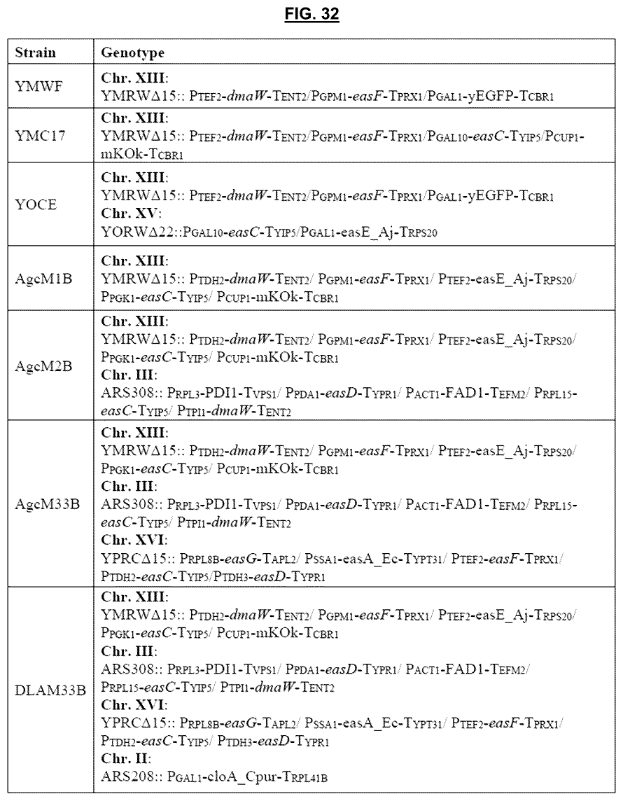

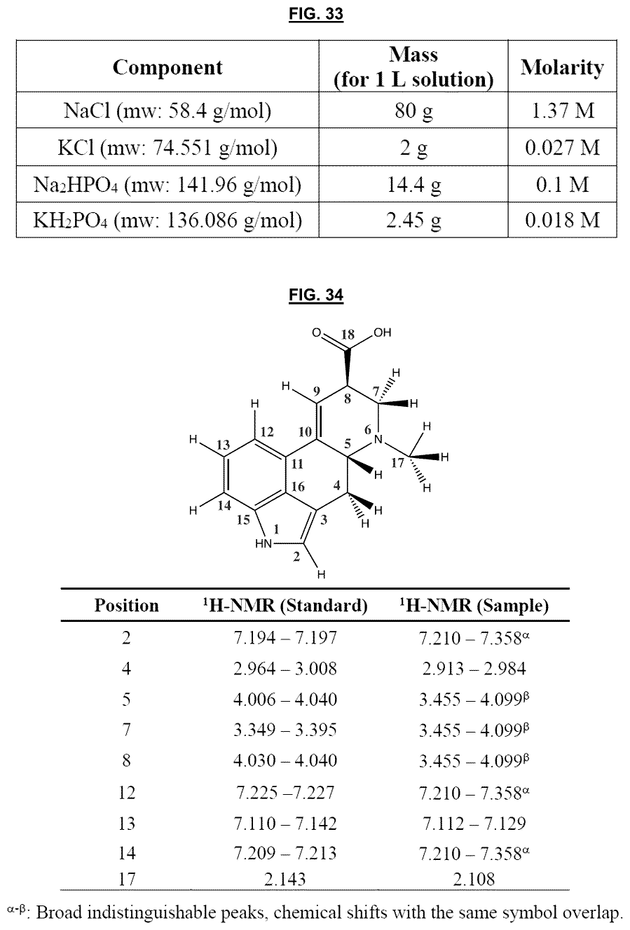

Directing the metabolic flux towards the agroclavine branch of the pathway, and thereafter DLA, requires the isomerase variant of EasA. As before, an SSN of EasA was generated, with the sequence from C. purpurea, to identify an isomerase isofunctional cluster. An earlier publication by Cheng, J. Z., et. al. (2010), reported the importance of the F176 residue, four sequences from this cluster with the structurally corresponding F176 residue were then selected and synthesized. These orthologues were then screened by co-expression with easD and easG in a strain with dmaW, easF, easC, and easE_Aj integrated in the yeast genome (YOCE; FIG. 32).

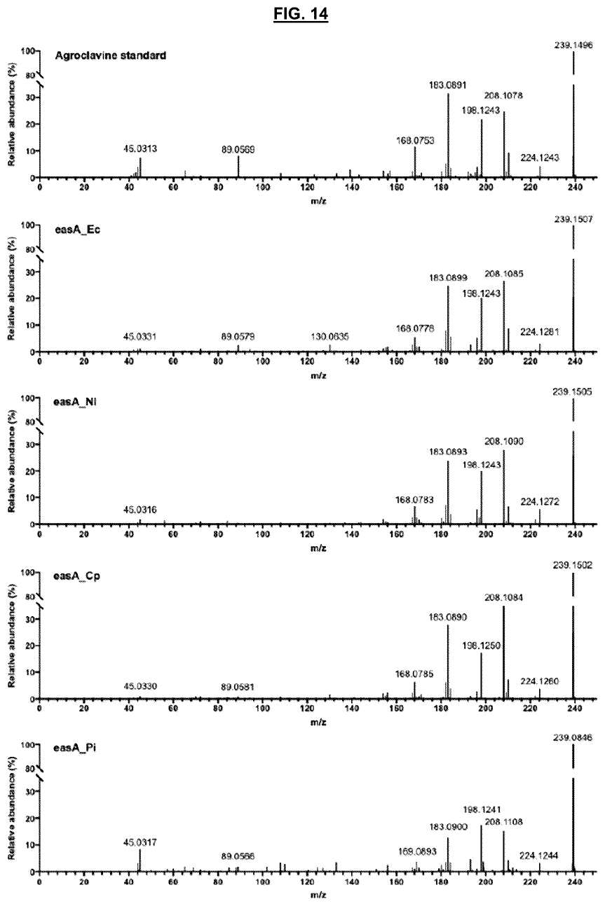

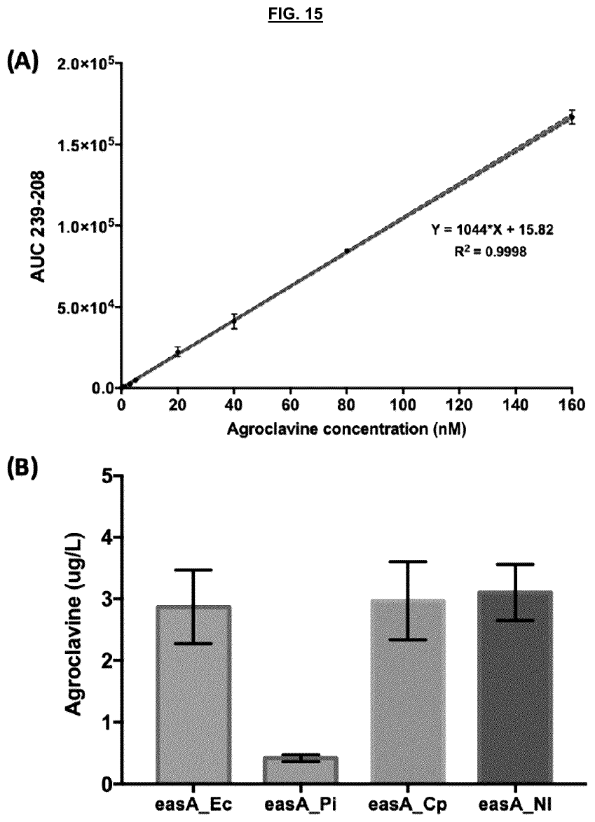

All easA orthologues produced a compound with a [M+H]⁺ of 239 m/z, corresponding to the agroclavine standard (FIG. 3B). Further comparison of the MS/MS fragmentation spectra confirmed the identity of the eluted compound to be agroclavine (FIG. 14). The amounts of agroclavine produced by the strains expressing the different EasA orthologues showed EasA_Ec, EasA_Cp, and EasA_Nl, were all producing approximately 2.8-3.1 μg/L, while the strain containing the EasA_Pi orthologue was producing around seven times less agroclavine (FIG. 15), and none of the selected EasA orthologs were found to be producing any of the alternative products. These results identified three equally suitable EasA candidates that could be used to build the DLA pathway; for simplicity down the line EasA_Ec was selected for further pathway construction.

### Example 4: Screening for a Functional Agroclavine Oxidase to Produce DLA

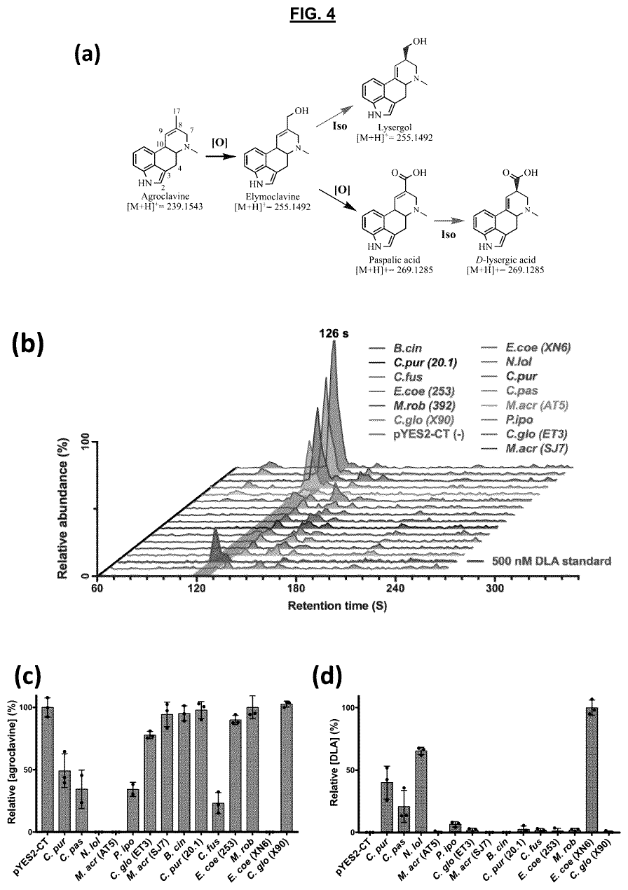

To complete the DLA biosynthetic pathway, the oxidation of agroclavine to DLA was addressed. This two-step oxidation was proposed to be catalyzed by a cytochrome P450 (CYP450) monooxygenase, clavine oxidase (CloA) (Haarmann, T., et al., ChemBioChem 7, 645-652 (2006)), though the mechanism of the reaction has yet to be biochemically characterized. From the myriad of ergot alkaloid producing fungi, a diverse set of oxidized agroclavine products have been isolated. These products correspond to products that have undergone either a single oxidation (elymoclavine/lysergol: [M+H]⁺=255 m/z) or a double oxidation (paspalic acid/DLA: [M+H]⁺=269 m/z) with the possibility of an isomerization of the C8-C9 double bond (elymoclavine/paspalic acid) to the C9-C10 position (lysergol/DLA) (FIG. 4A).

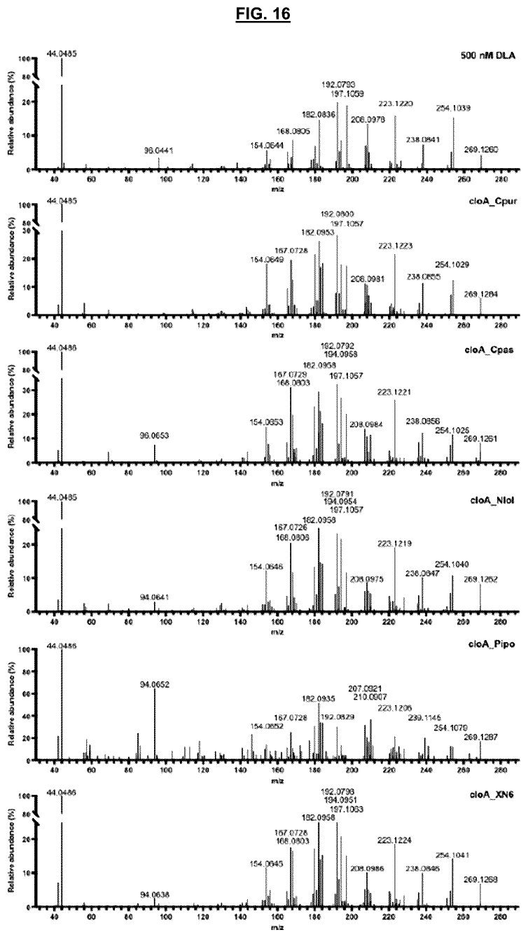

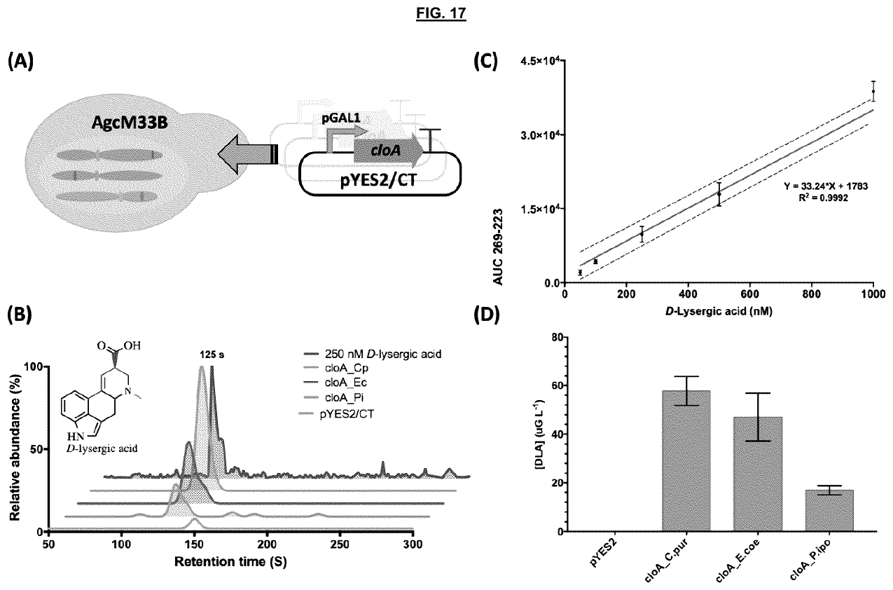

Identification of an orthologue that expresses in yeast and specifically produces DLA was then carried out. With an SSN generated from the cloA sequence predicted from C. purpurea, 15 orthologues were identified from a cluster that consisted of sequences from organisms where DLA has been isolated from. These 15 orthologues were screened for functional expression in yeast (FIG. 4B). When provided with the agroclavine substrate, 5 of the 15 orthologues were found to catalyze the production of a molecule with a [M+H]⁺=269 m/z that shared the same retention time as the DLA standard (FIG. 4c). The molecule also had identical MS/MS fragmentation spectra as a DLA standard, confirming that the molecule was DLA (FIG. 16).

This screen has thus identified five orthologues of cloA (C. pur., C. pas., N. lol., E. coe, P. ipo.) that could be used for pathway construction. The top two producers (C. pur., and E. coe.), and the worst producer (P. ipo.) from this screen (FIG. 4) were then further tested for DLA production in the context of an agroclavine-producing host (FIGS. 17A, 17B). The C. purpurea and E. coenophialia orthologues were found to produce comparable levels of DLA in this context (FIG. 17D) and the C. purpurea orthologue was selected for incorporation into the modified strain due to lower variability in DLA titers.

### Example 5: Assembling the Components of the Complete DLA Biosynthetic Pathway in Yeast

Equipped with a functional set of enzymatic and genetic elements for the reconstitution of the DLA biosynthetic pathway in yeast, a prototype DLA-biosynthetic strain was sought to be constructed. The initial prototype was modelled after the strain that had been developed for the production of cycloclavine (Jakubczyk, D., et al., Angewandte Chemie International Edition 54, 5117-5121 (2015)) that reported achieving an admirable yield of 529 mg L⁻¹. The present prototype design used stronger promoters for the less functional enzymes, such as easE, and multiple copies of the other pathway enzymes driven by weaker promoters in an attempt to attenuate the effects metabolic burden. Additional copies of the genes FAD1 and PDI1 from yeast were also included, which have been shown to aid protein folding and enhance the production of flavin adenine dinucleotide (FAD), a key co-factor for EasE activity (Nielsen, C. A., et al., Microbial cell factories 13, 1 (2014)).

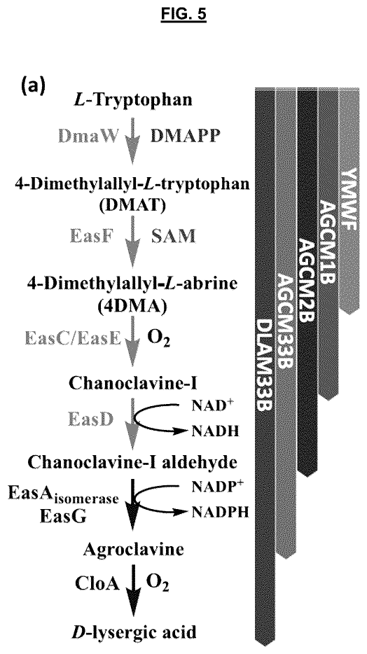

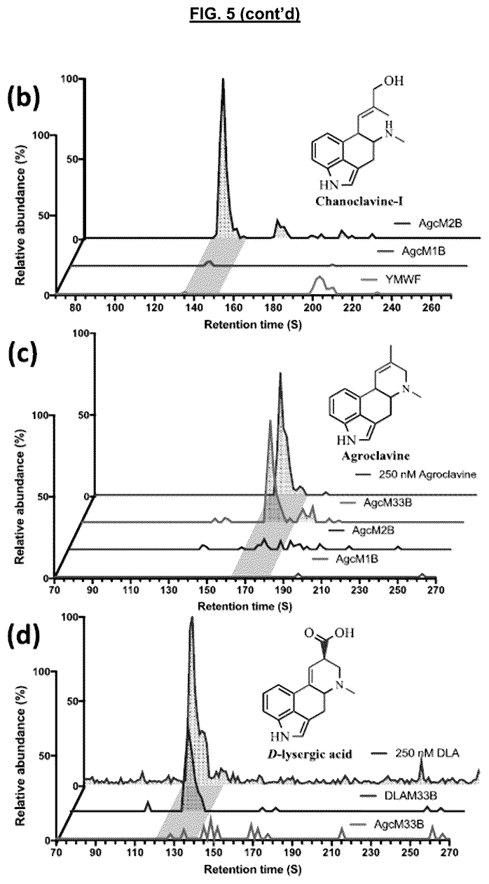

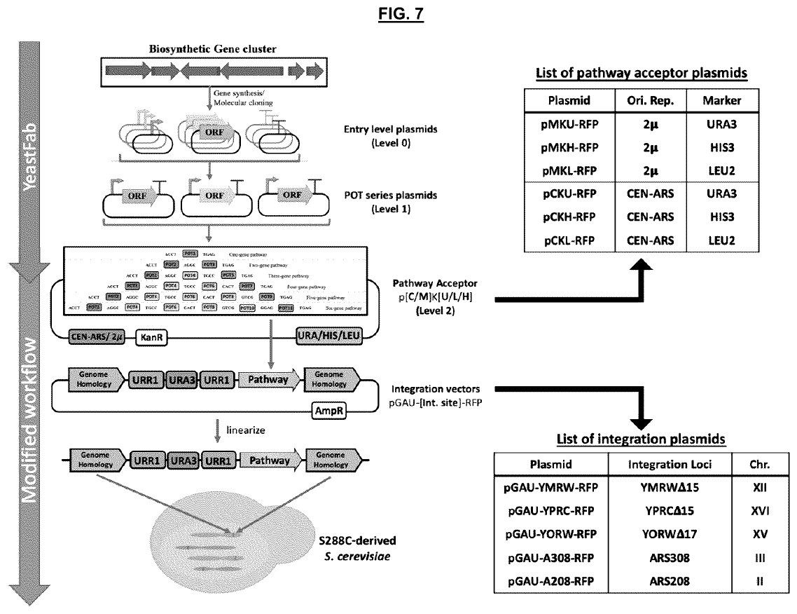

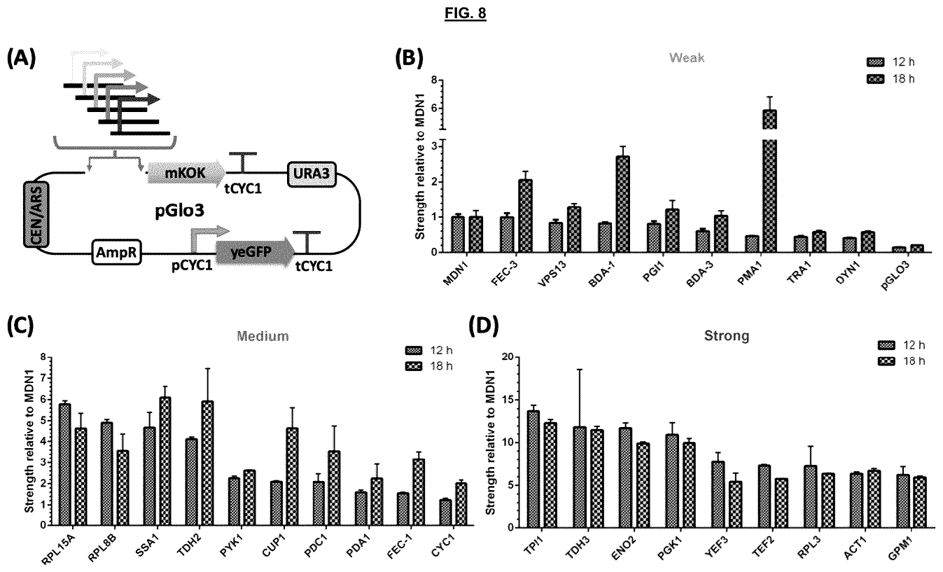

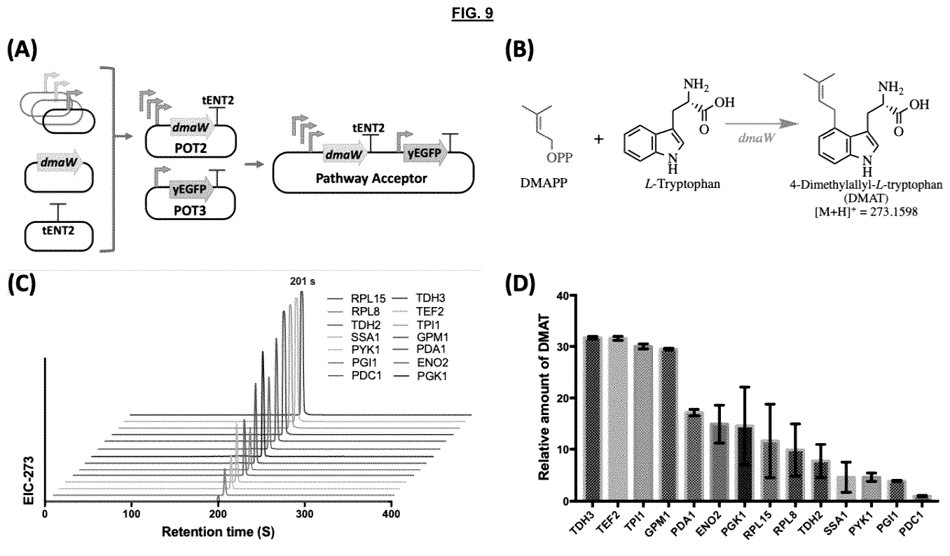

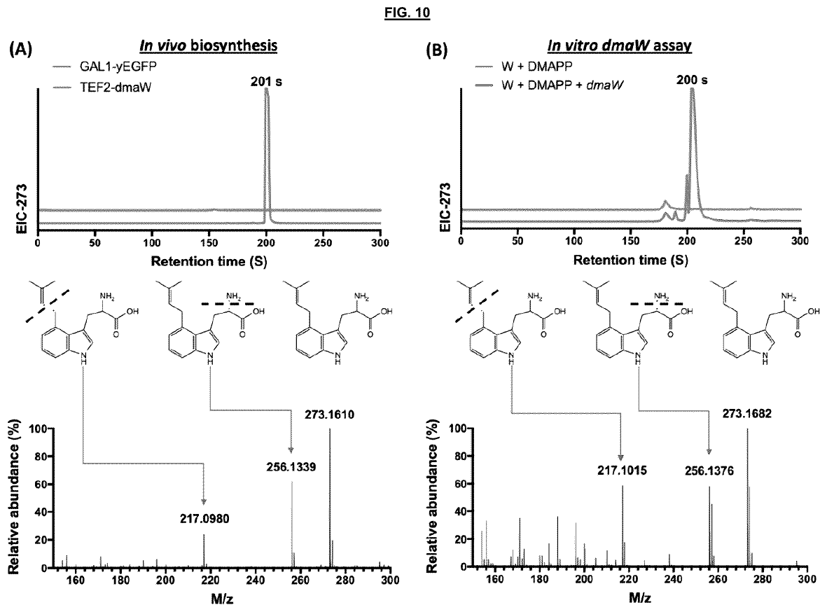

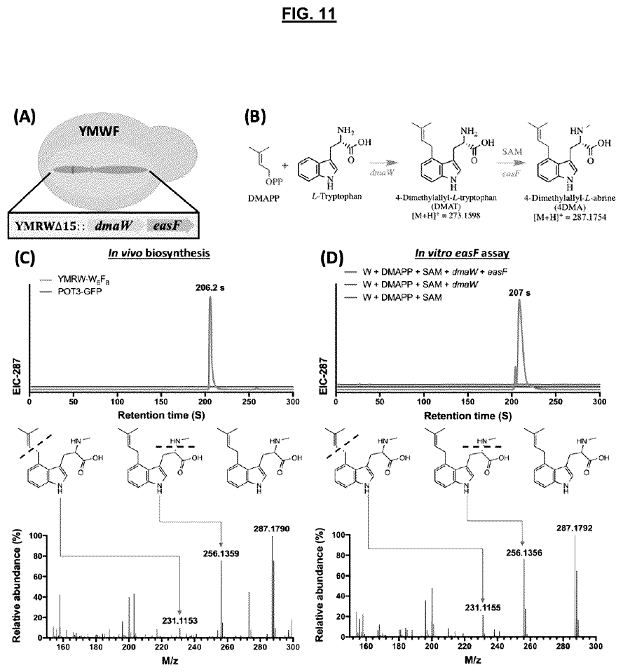

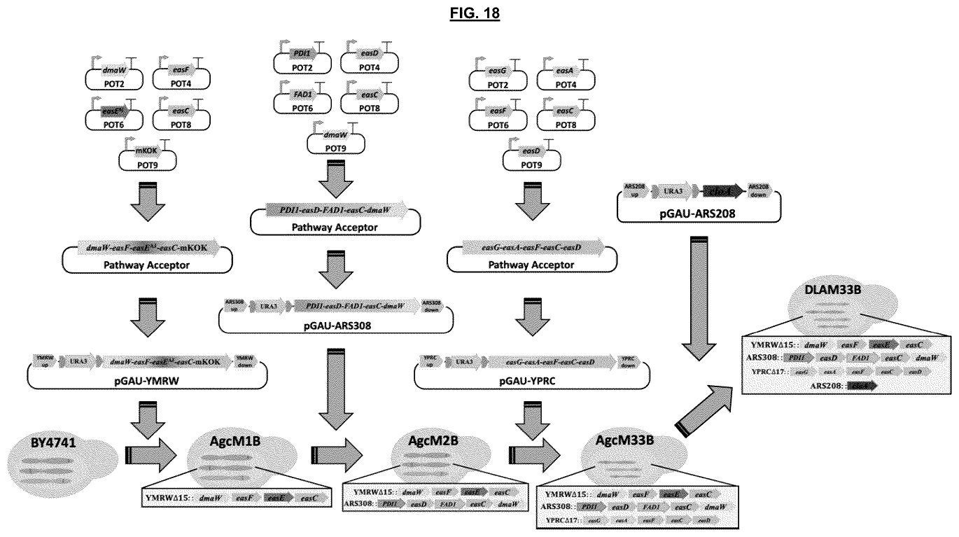

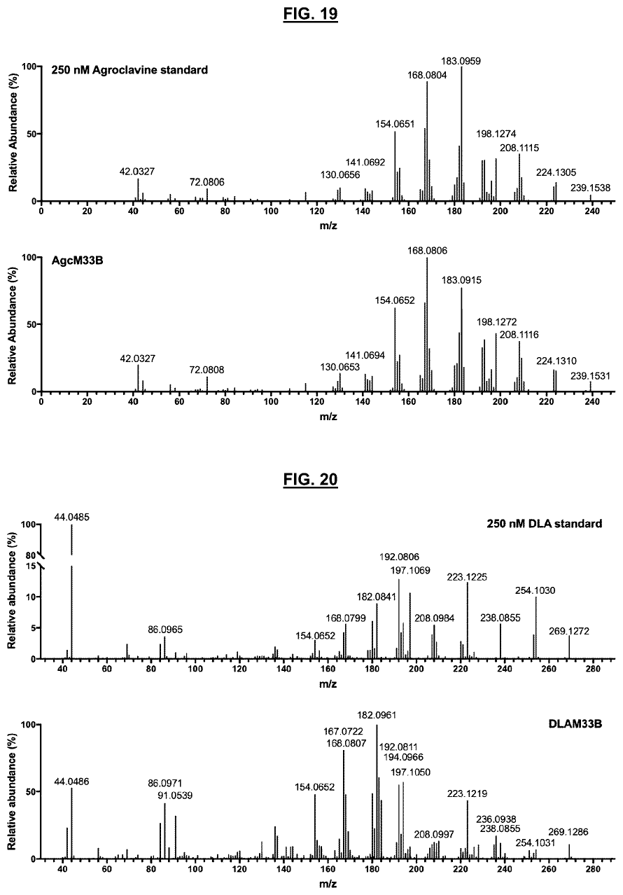

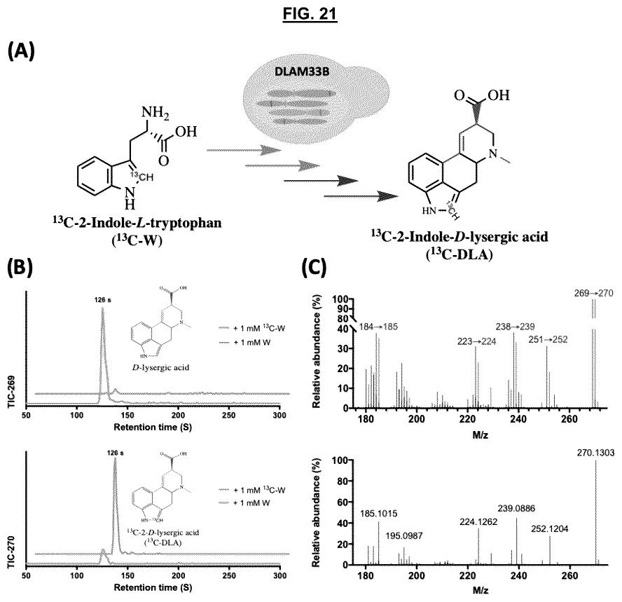

Following the modified YeastFab pipeline (FIG. 7), the prototype strain was sequentially constructed and expanded as four segments (FIG. 18). Each segment was designed to produce intermediate products along the DLA biosynthetic route that can be easily detectable, namely chanoclavine-I, agroclavine, and DLA. This approach facilitated troubleshooting as well as enabling each intermediate strain to serve as a negative control for subsequent strains. Introduction of the first two segments (AgcM1B, AgcM2B; FIG. 32) containing the genes for the early ergot pathway resulted in the production of chanoclavine-I (FIG. 5A). Increasing the copy numbers of easC and dmaW, as well as the supplemental expression of FAD1 and PDI1, improved chanoclavine-I yields as reported previously (Nielsen, C. A., et al., Microbial cell factories 13, 1 (2014)). In the third segment, easG, easA_Ec, and further additional copies of easF, easC, and easD were introduced. This strain, AgcM33B, with three segments integrated into the yeast genome, produced a compound with a [M+H]⁺=239 m/z, that co-elutes with the agroclavine standard (FIG. 5B) and confirmed to be agroclavine by MS/MS fragmentation (FIG. 19). The final reaction to DLA was then incorporated by the integration of cloA_Cpur as the last segment into the ARS208 site. LC-MS analysis of the spent media from this strain (DLAM33B; FIG. 32) showed the production of a compound with a [M+H]⁺=269 m/z, that co-eluted with the DLA standard (FIG. 5C) and was further confirmed by MS/MS fragmentation (FIG. 20).

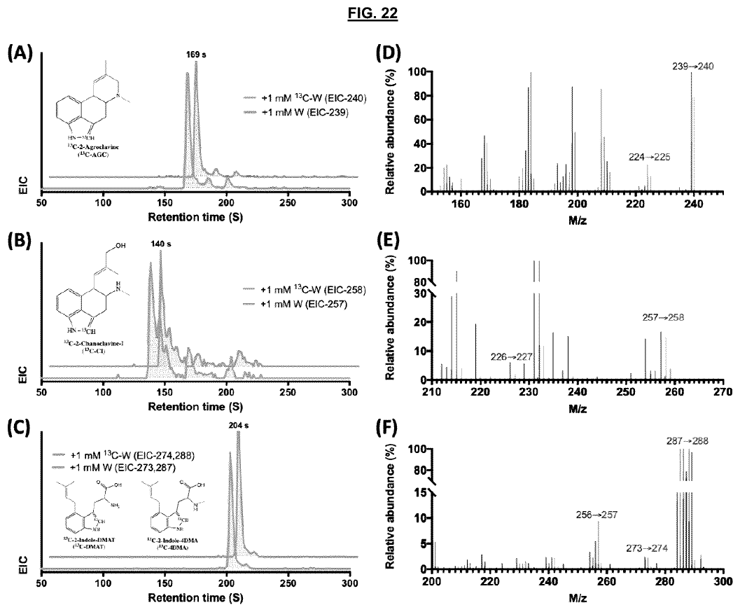

Next, the reconstitution of the pathway producing DLA was validated by supplying the present DLAM33B strain with ¹³C-2-indole-L-tryptophan (¹³C-W) as a feedstock (FIG. 21A), to track the progression of tryptophan through the reconstituted pathway. In these experiments, a shift of 1 m/z was detected in the chromatogram peak corresponding to DLA and in greater abundance than expected for naturally occurring ¹³C-DLA (FIG. 21B). The same mass shift was observed for all the intermediates of the DLA biosynthesis pathway (FIG. 22). These data corroborated with the expected [¹³C-M+H]⁺ values incorporating ¹³C-W into the pathway and subsequent turnover into ¹³C-DLA and its ¹³C-intermediates. A comparison of the mass spectral patterns for the proposed ¹³C-DLA further illustrated this +1/z shift across all dominant fragmentation peaks (FIG. 22C, top) and indicated the utilization of tryptophan to produce DLA in the introduced pathway. Considering that the base strain of S. cerevisiae used in this work is not tryptophan auxotrophic, this experiment indicates that the engineered strain can produce DLA from tryptophan derived from central metabolism or the culture media. The next quantum to validate the production of DLA is by NMR analysis. To achieve this, a scale-up of the shake-flask process was performed to obtain the material required. The chemical shifts of the concentrated extracts were assigned based on predicted values and compared against the DLA standard (FIG. 34).

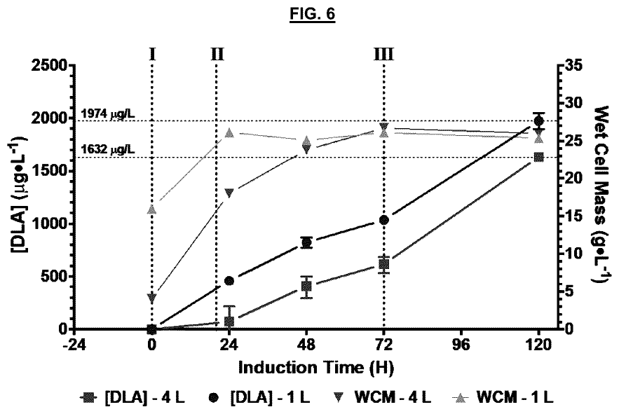

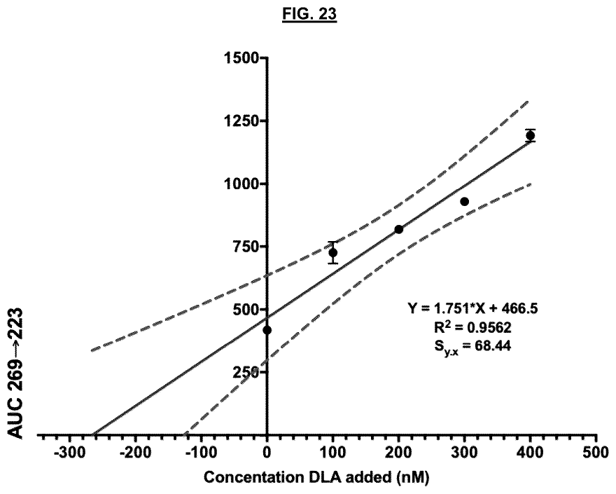

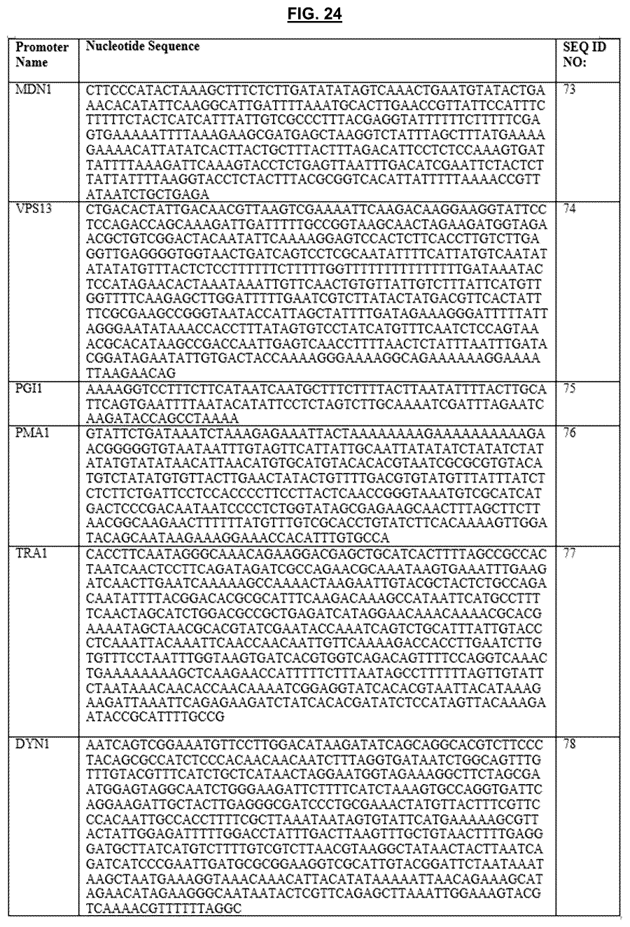

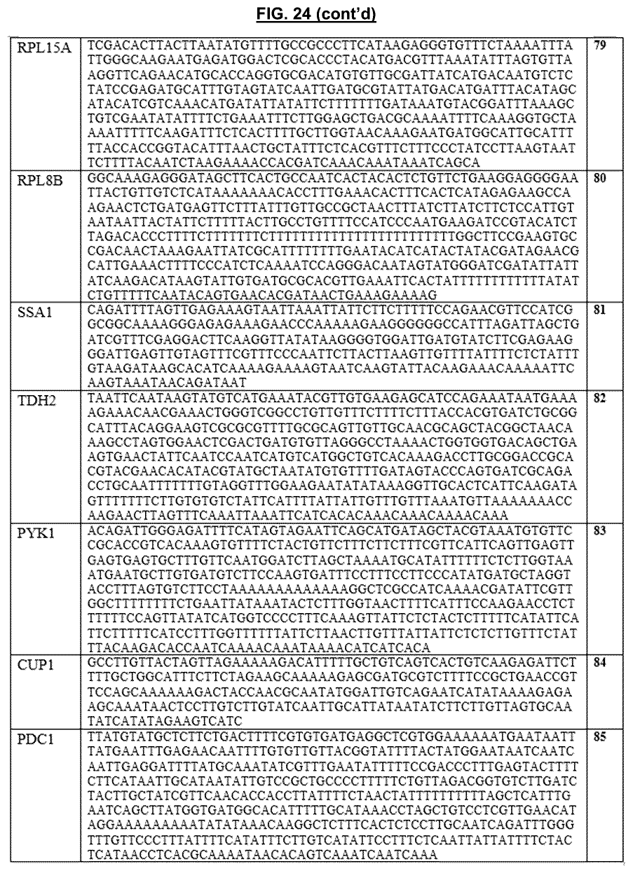

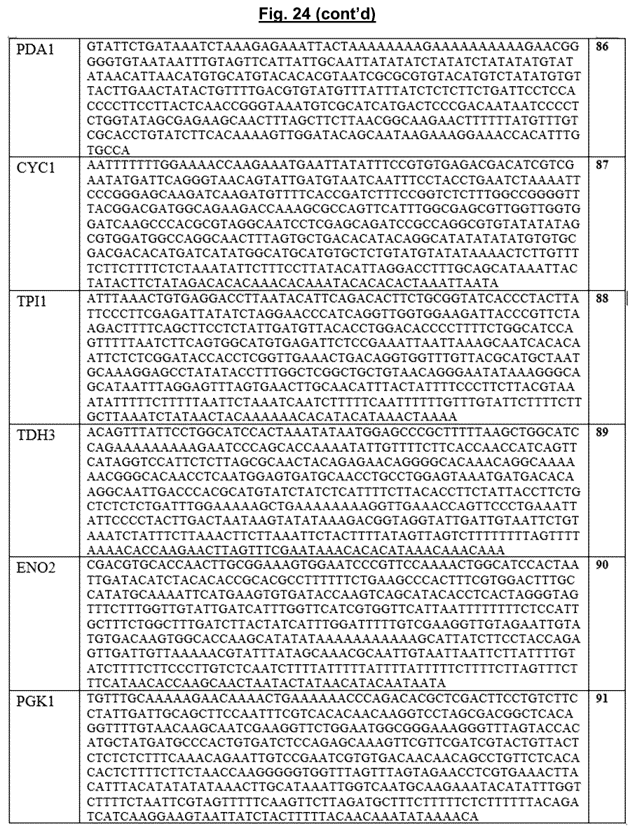

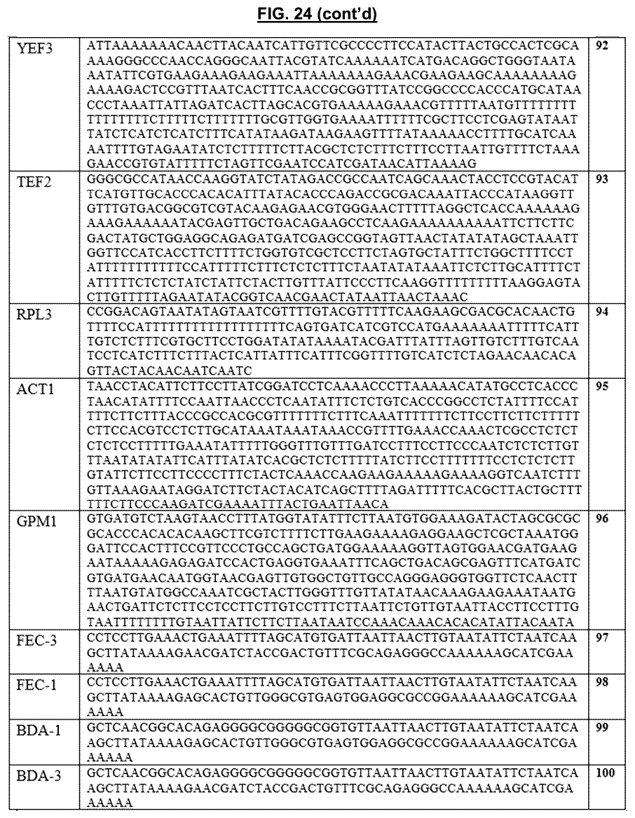

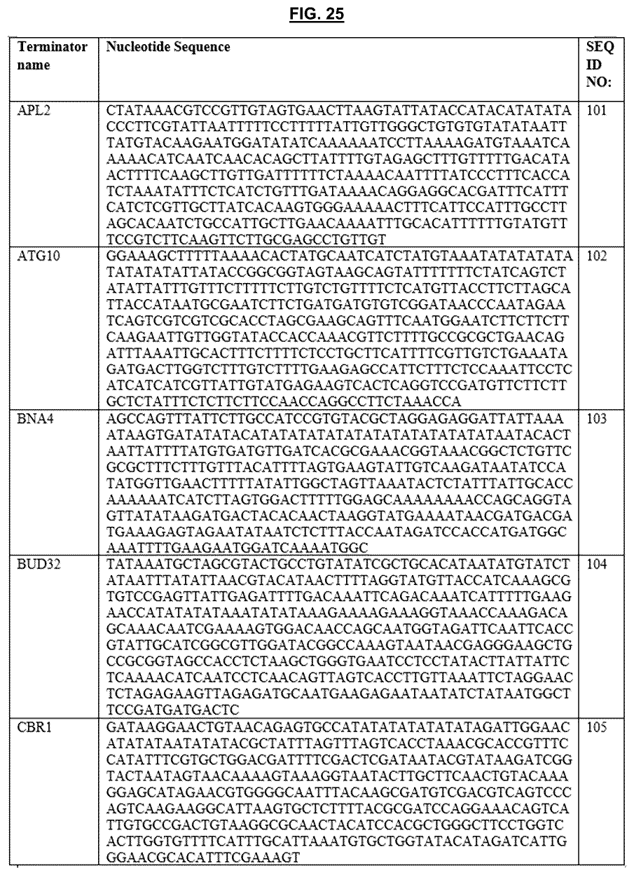

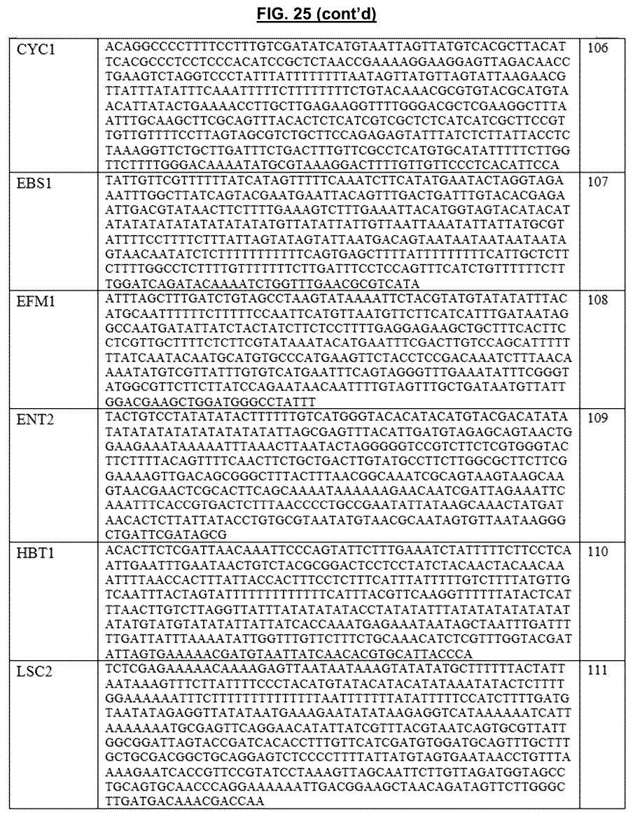

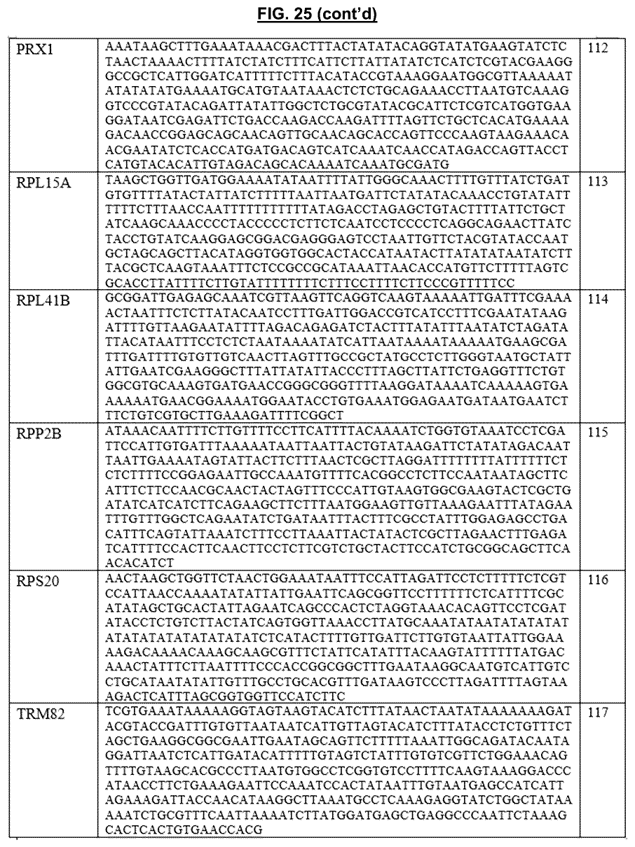

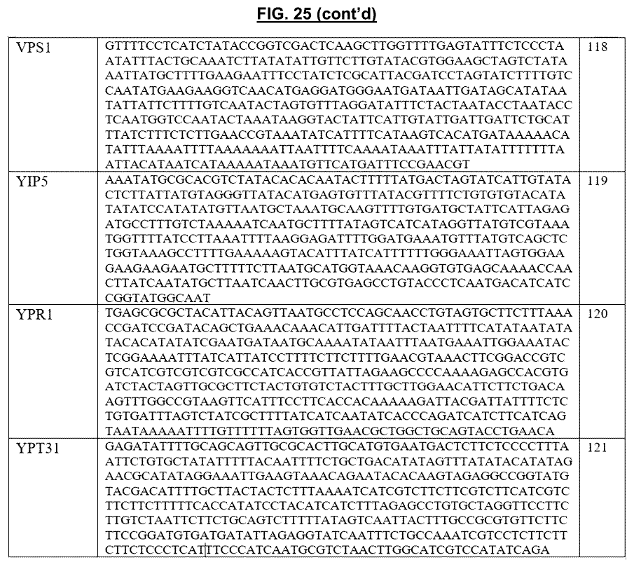

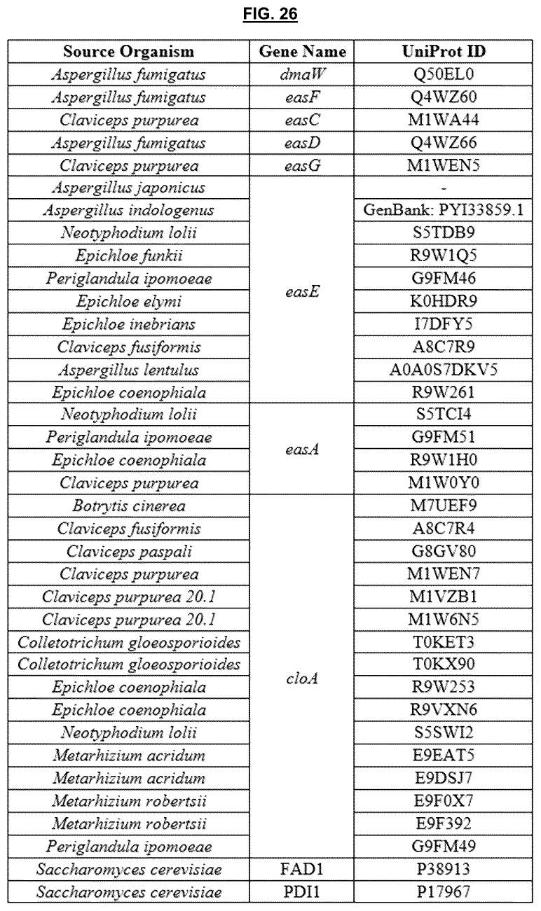

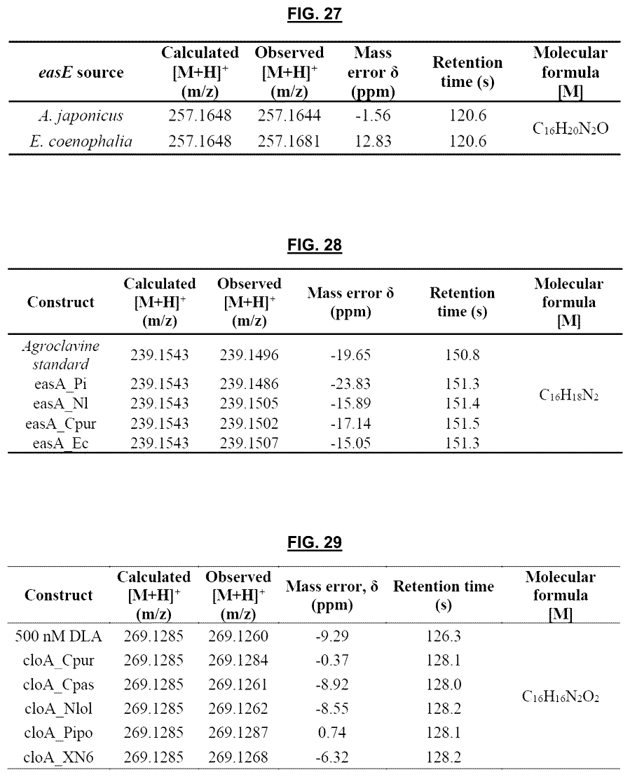

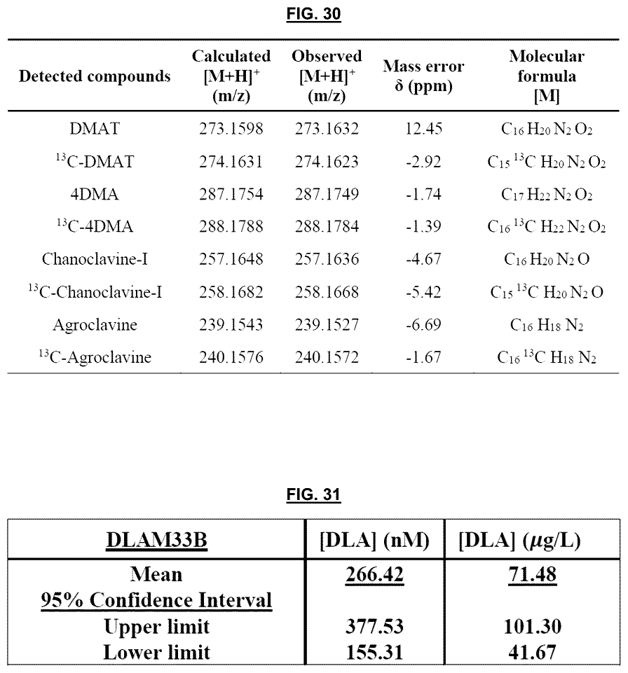

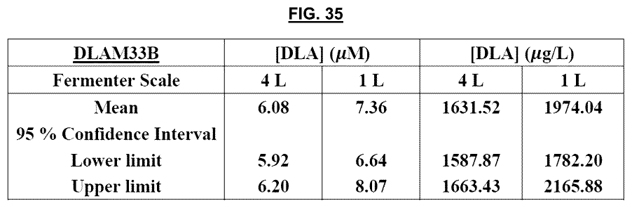

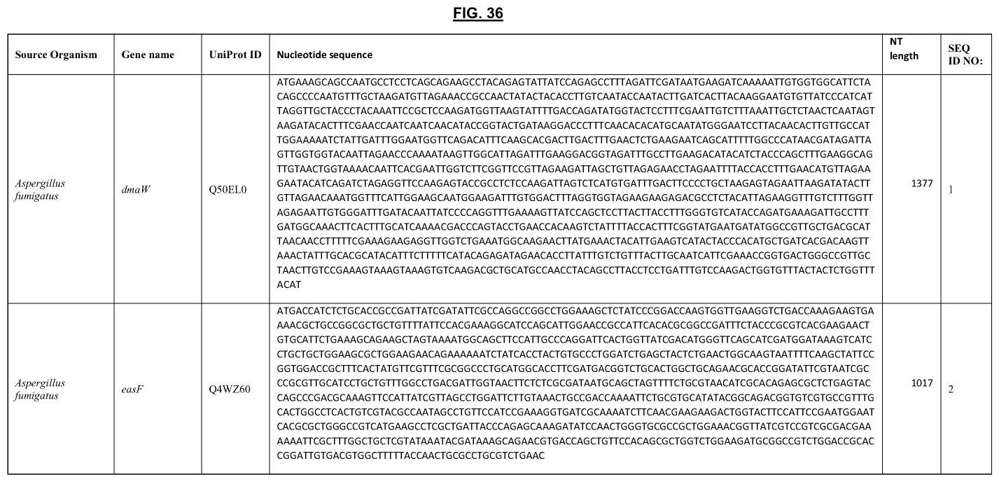

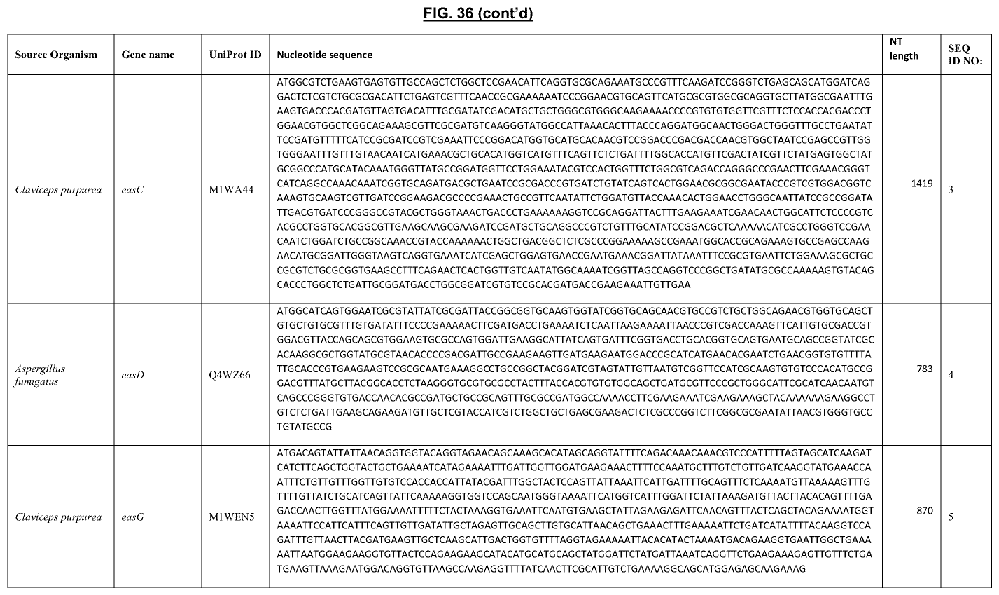

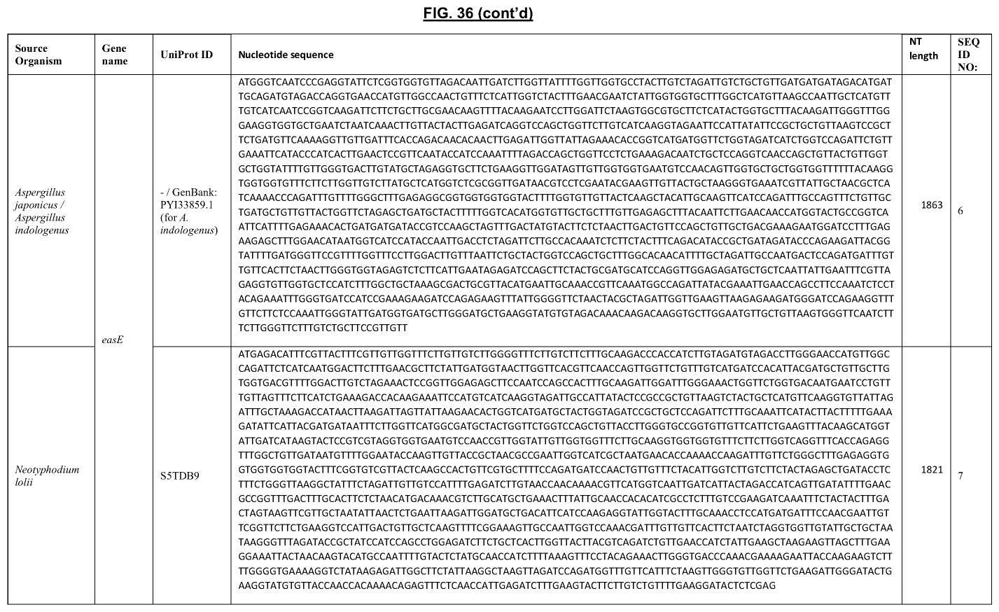

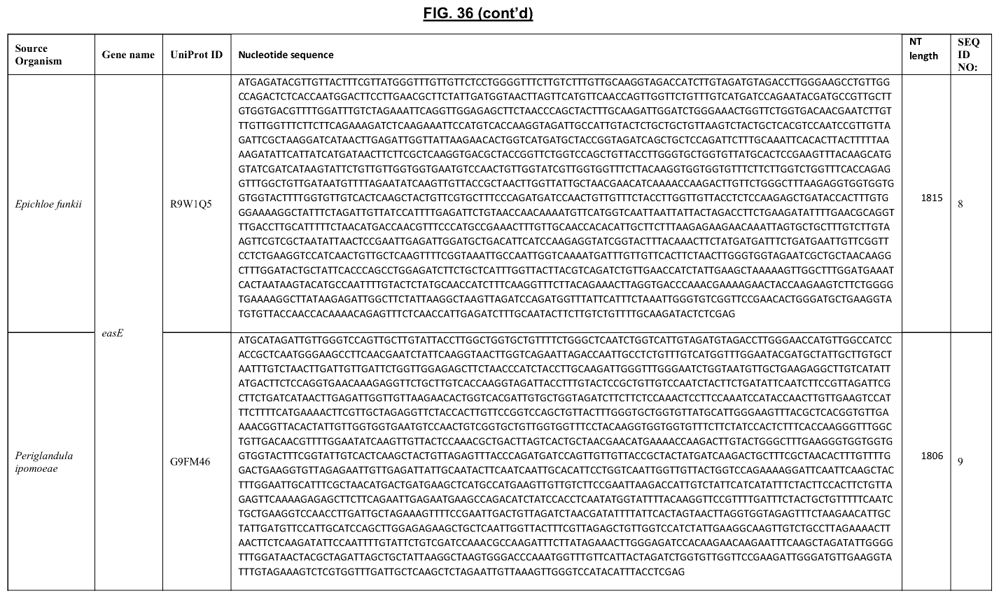

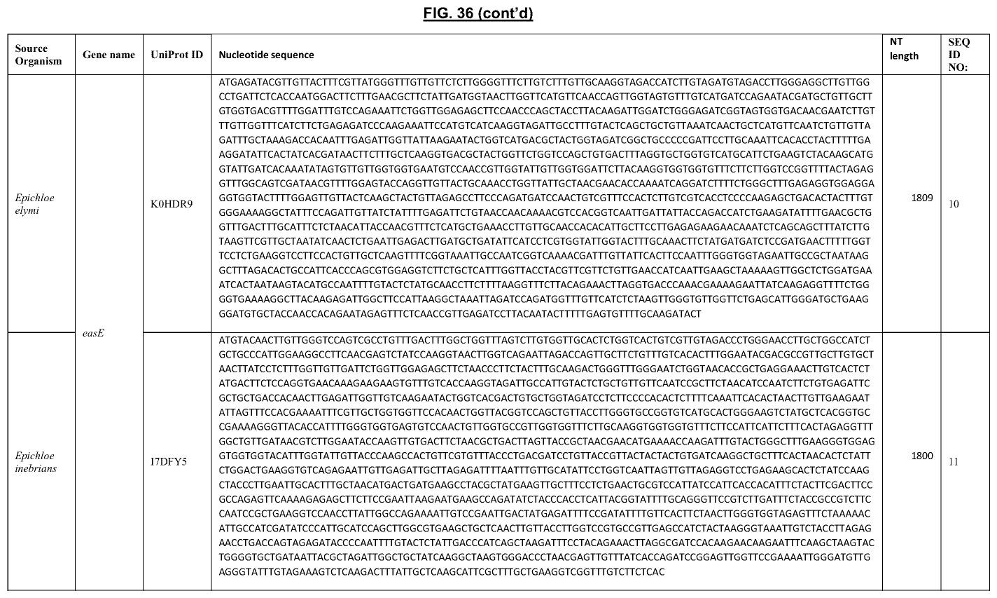

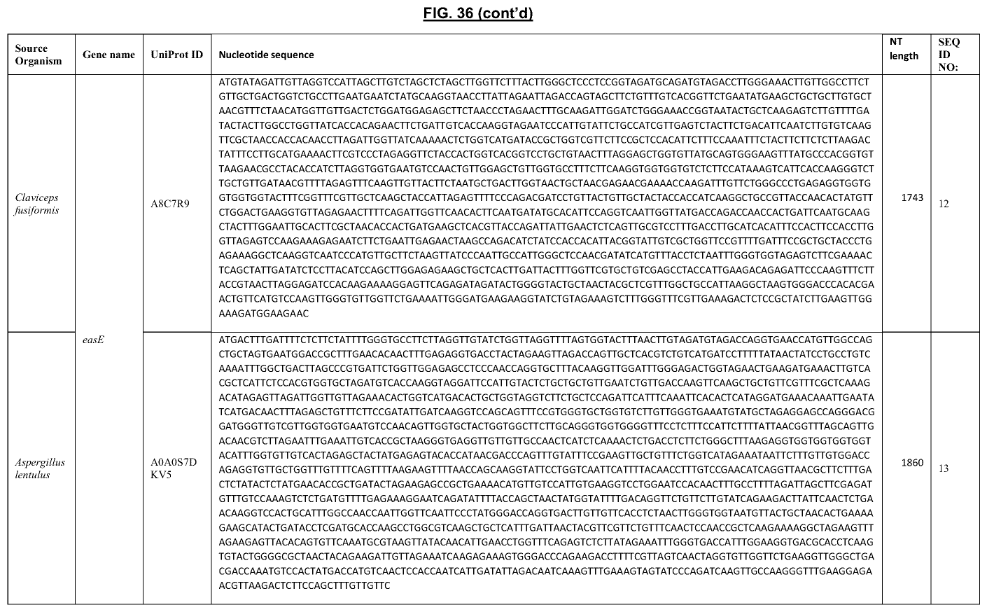

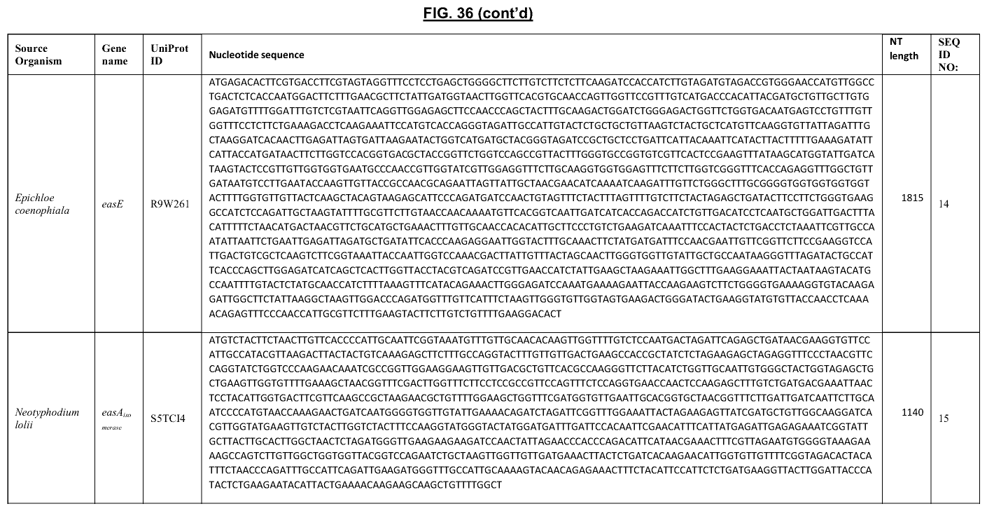

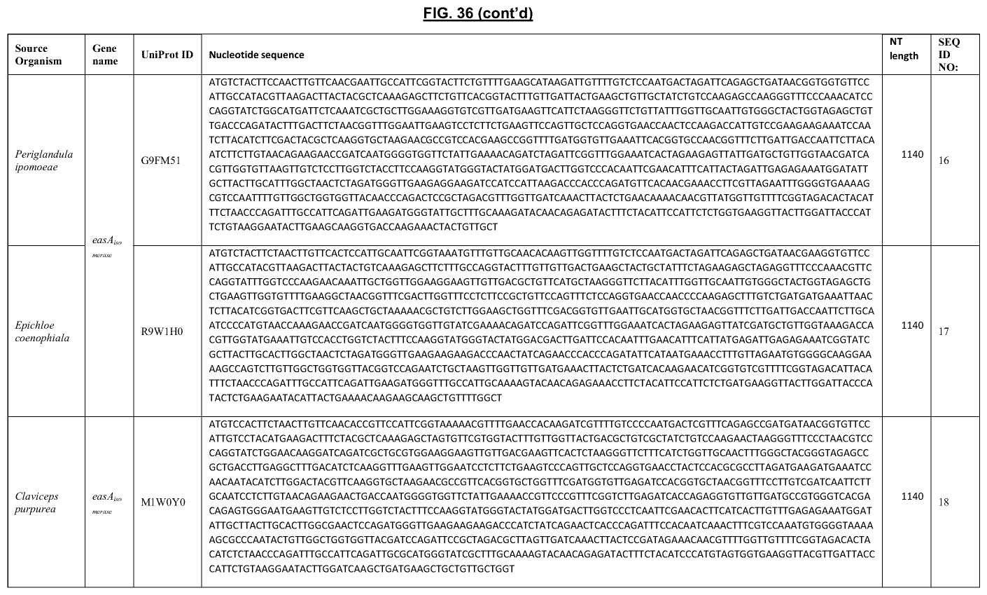

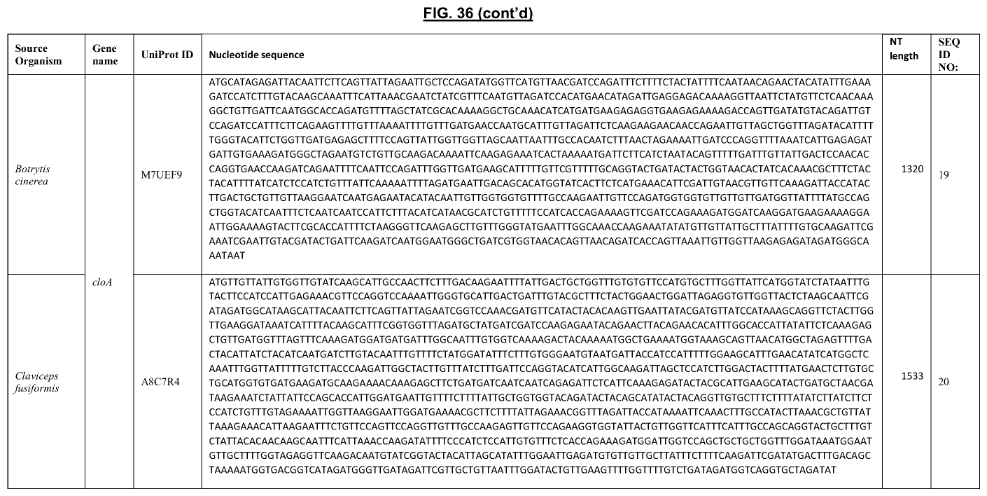

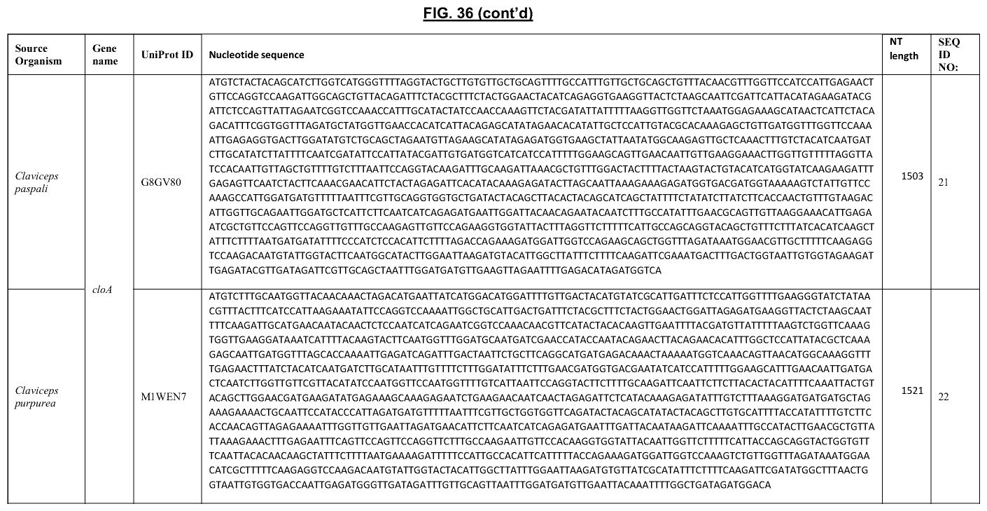

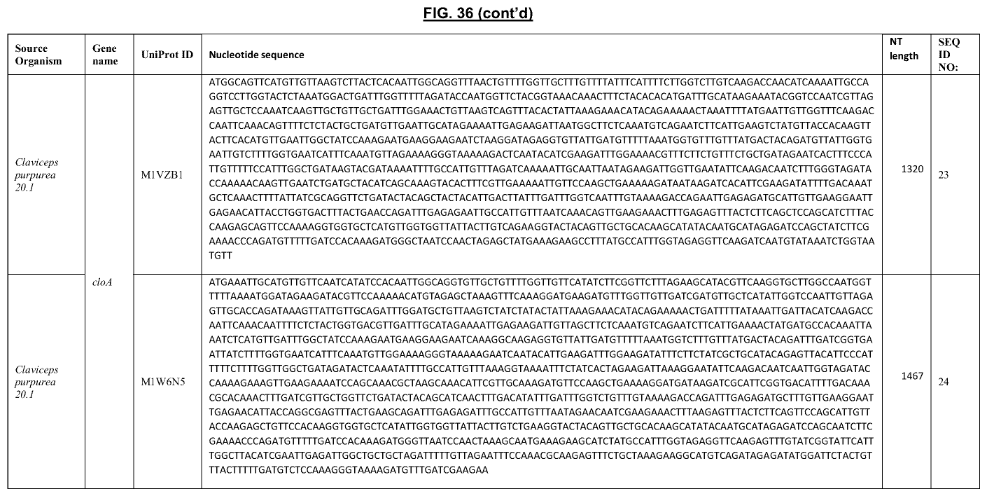

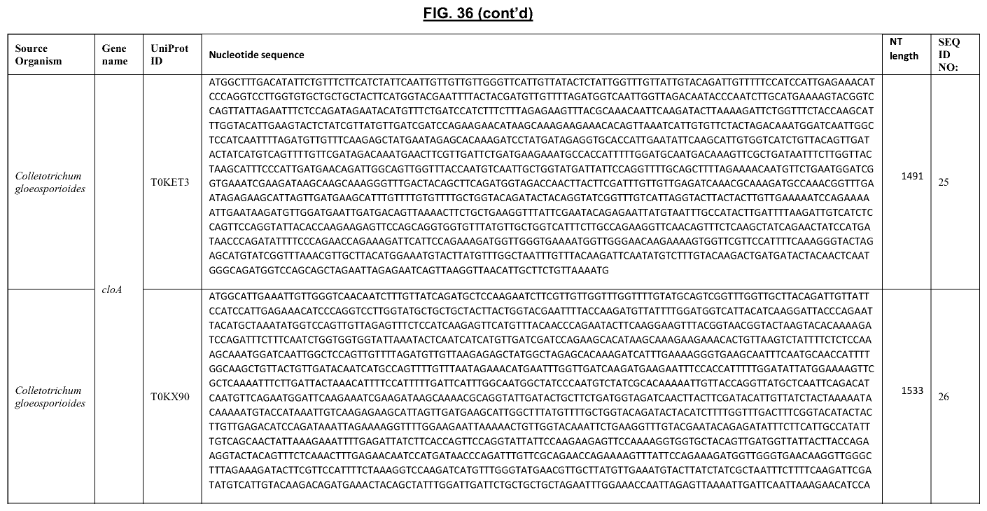

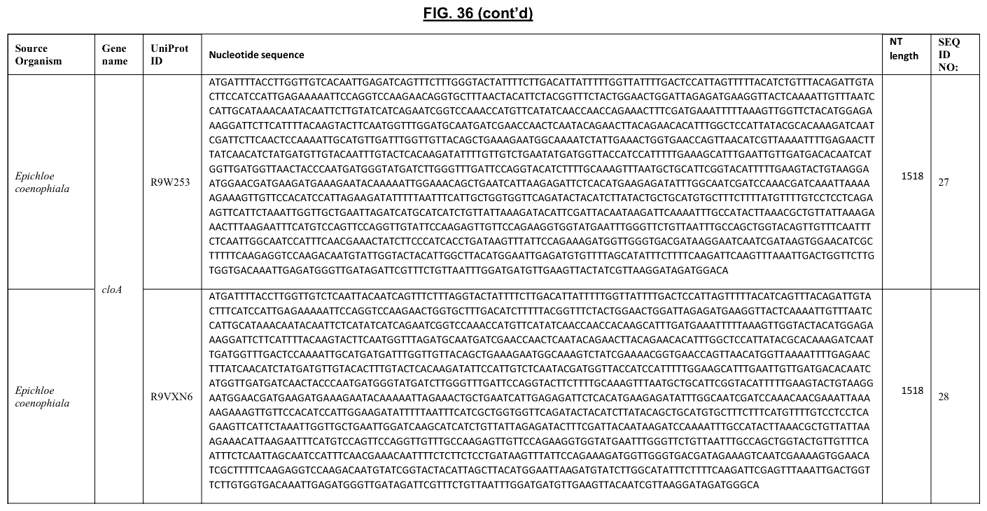

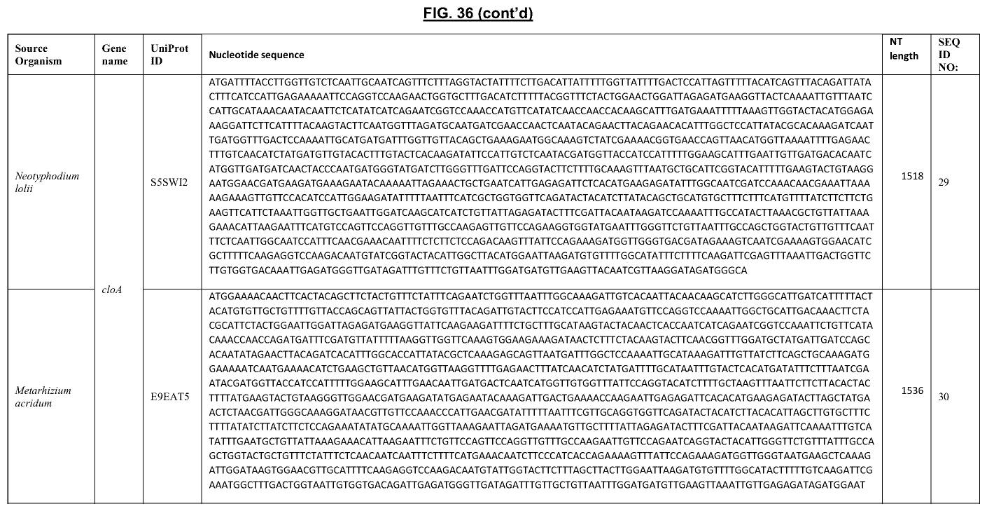

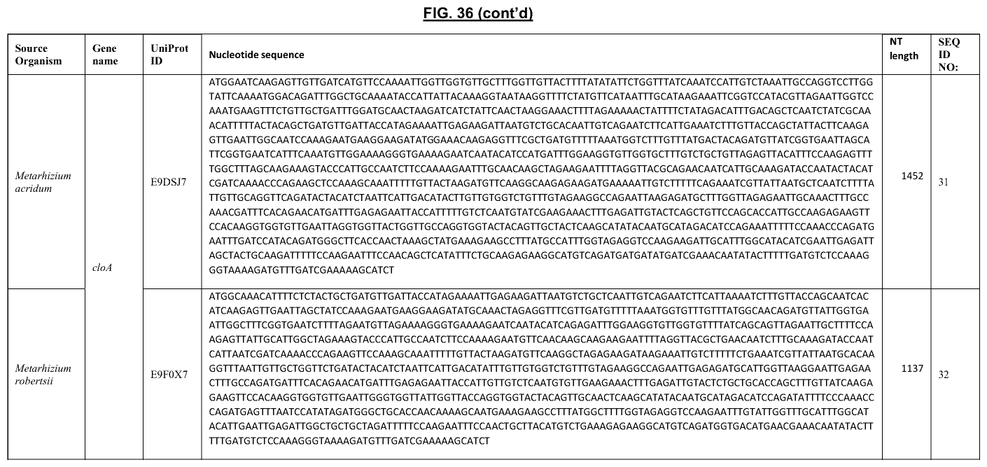

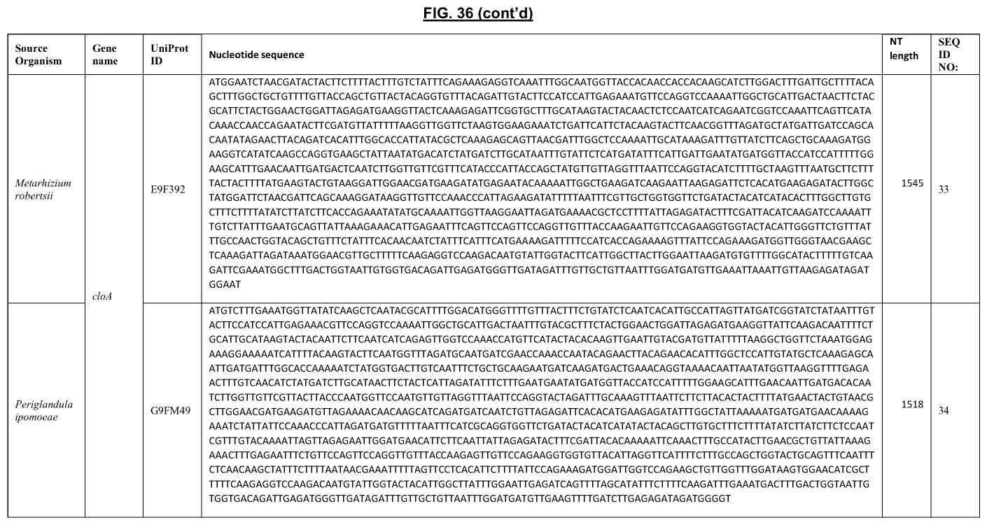

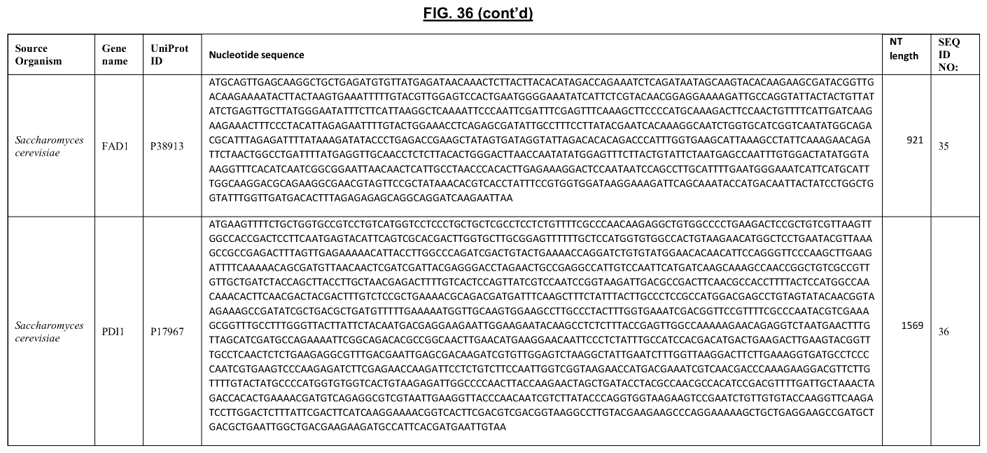

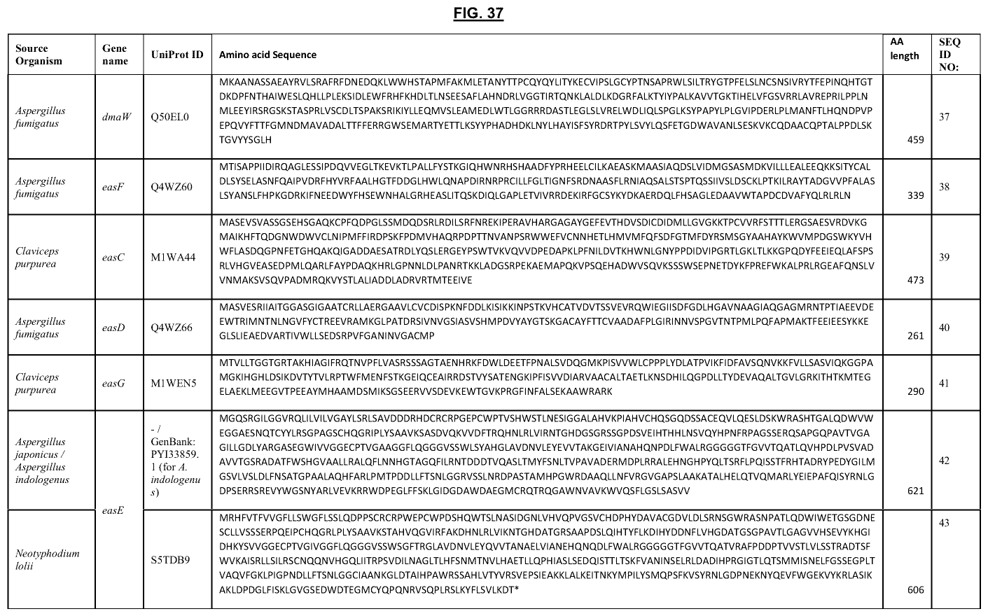

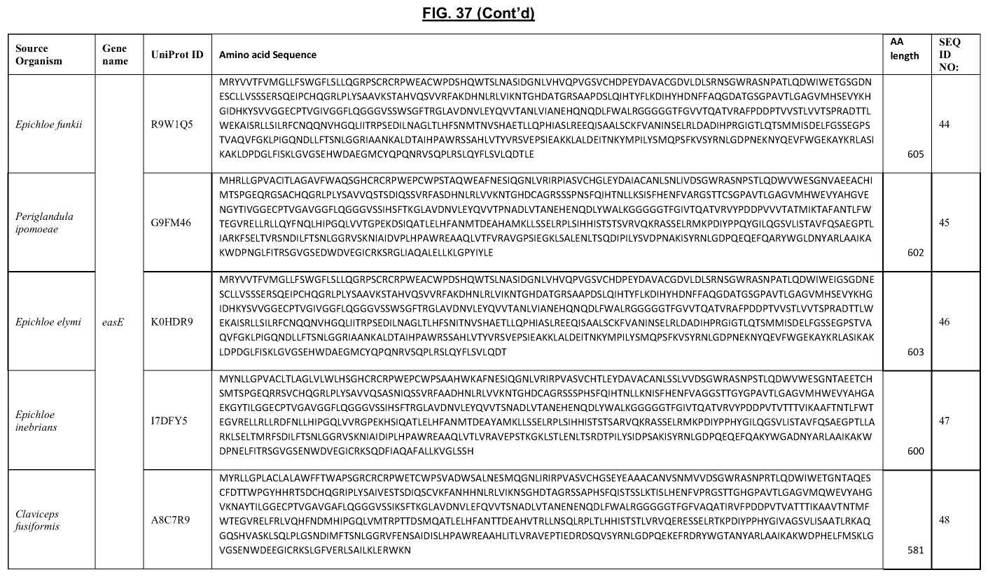

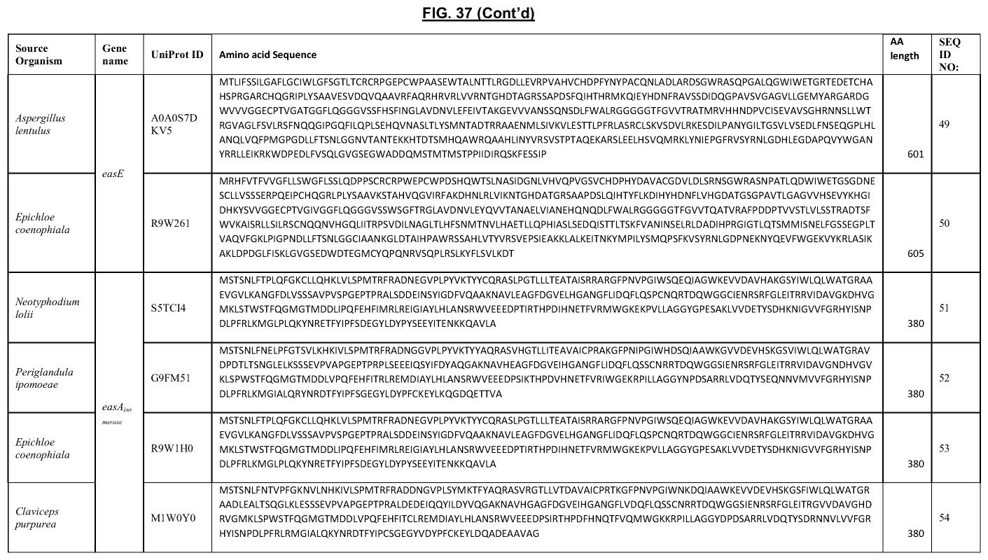

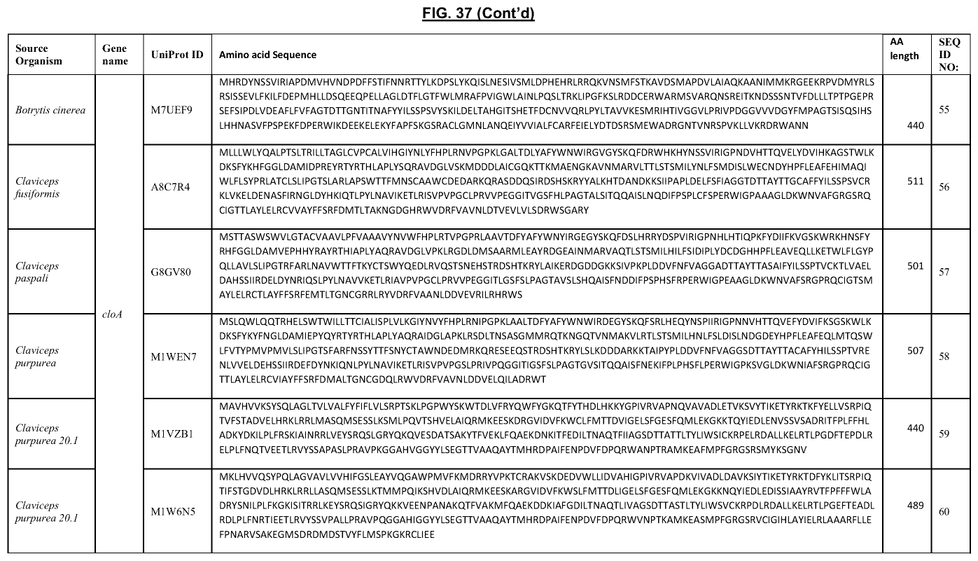

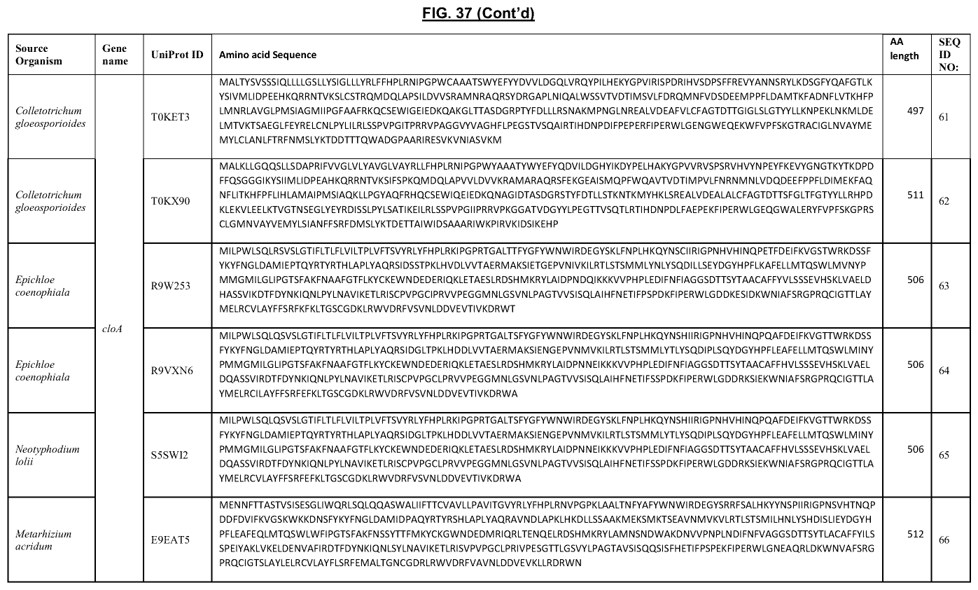

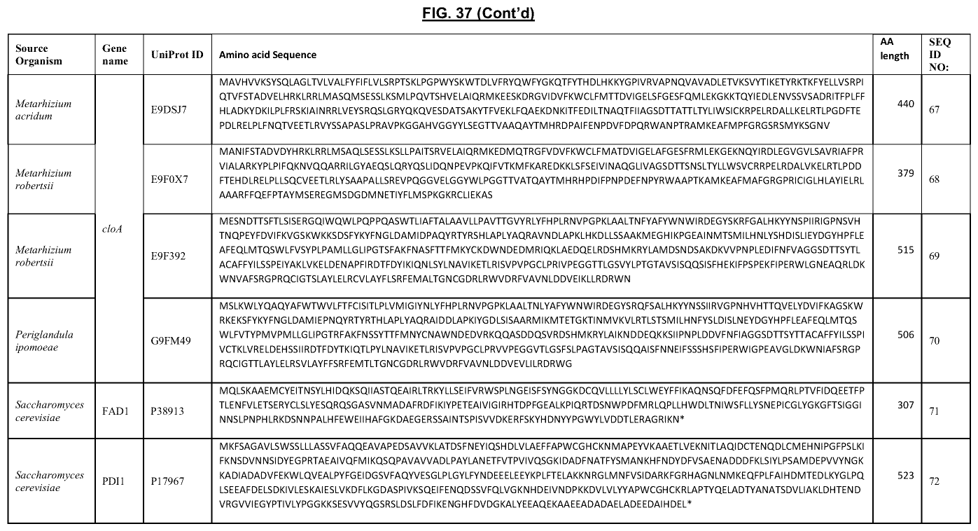

The endpoint production titre of DLAM33B strain in small scale shake-flask conditions measured to be 71.5 μg L⁻¹ (FIG. 23). To demonstrate the scalable application of the engineered strain, 1 and 4 L scale bioreactor fermentations were further attempted. In these fermentation experiments with the improvement of culture oxygenation, carbon source, and inducer control (galactose was used as both carbon source and expression induction agent), as well as pH control, a sustained cell proliferation to a maximum cell density of around 25-26 g L⁻¹ and improved production titers (FIG. 6), attaining 2.0 mg L⁻¹ and 1.6 mg L⁻¹ at the end points for the 1 and 4 L fermentations, respectively (FIG. 35) were achieved. It is therefore conceivable that with further bioprocessing and strain optimization, commercial level titres could be attainable.

### DISCUSSION

In this work, the identification of alternative orthologues of the fastidious enzymes along the ergot alkaloid pathway using the SSN generated from the EFI-EST webtool was described, and along with it the development of customized strains for their systematic screening. Through this approach, central to the tenets of synthetic biology, candidates for the reconstitution of the pathway to DLA in S. cerevisiae were successfully identified. These were subsequently used to create an engineered yeast strain capable of producing DLA from central metabolism. To the best of the inventors' current knowledge, this is the first demonstration of the heterologous total biosynthesis of DLA from simple sugar. Advantageously, the provision of an engineered microbe capable of producing lysergic acid directly from simple sugars and amenable towards commercial scale fermentation may reduce production costs. Further strain optimization, aimed towards identifying and relieving bottlenecks in the pathway, as well as improved bioprocess optimization, would indubitably push production titres towards required commercial levels.

This work builds on the growing body of work demonstrating the use of industrially tractable microorganisms for the production of complex natural products using economical and renewable feedstock, such as what has been done for the biosynthesis of the opioids (Galanie, S., et al., Science 349, 1095-1100 (2015)). Engineered strains provide an excellent platform to drive the discovery of semi-synthetic therapeutic lead compounds, as well as for developing pilot strains for producing important naturally derived therapeutics.

Lastly, with the recent renaissance of research into repurposing psychedelic compounds for anti-depressives and anti-anxiolytics applications (De Gregorio, D., et al., Elsevier Vol. 242 69-96 (2018)), it is believed that the present recombinant strain as described could be used to support efforts to probe the natural and semi-synthetic chemical space of ergoline-based therapeutics, to identify leads with enhanced therapeutic potential and fewer adverse effects.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

Any listing or discussion of an apparently prior-published document in this specification should not necessarily be taken as an acknowledgement that such document is part of the state of the art or is common general knowledge.
Chen, J.-J., Han, M.-Y., Gong, T., Yang, J.-L. & Zhu, P. Recent progress in ergot alkaloid research. RSC Adv. 7, 27384-27396 (2017).
Cheng, J. Z., Coyle, C. M., Panaccione, D. G. & O'Connor, S. E. A role for old yellow enzyme in ergot alkaloid biosynthesis. J. Am. Chem. Soc. 132, 1776-1777 (2010).
Cheng, J. Z., Coyle, C. M., Panaccione, D. G. & O'Connor, S. E. Controlling a structural branch point in ergot alkaloid biosynthesis. J. Am. Chem. Soc. 132, 12835-12837 (2010).
Cvak, L. In Ergot: the genus Claviceps 373 (CRC Press, 1999).
De Gregorio, D., Enns, J. P., Nuñez, N. A., Posa, L. & Gobbi, G. In Progress in brain research, 242 69-96 (Elsevier, 2018).
de Groot, A. N., van Dongen, P. W., Vree, T. B., Hekster, Y. A. & van Roosmalen, J. Ergot alkaloids. Drugs 56, 523-535 (1998).
Engler, C., Gruetzner, R., Kandzia, R. & Marillonnet, S. Golden gate shuffling: a one-pot DNA shuffling method based on type IIs restriction enzymes. PloS one 4, e5553 (2009).
Floss, H. G. et al. Biosynthesis of ergot alkaloids. Evidence for two isomerizations in the isoprenoid moiety during the formation of tetracyclic ergolines. J. Am. Chem. Soc. 90, 6500-6507 (1968).
Galanie, S., Thodey, K., Trenchard, I. J., Interrante, M. F. & Smolke, C. D. Complete biosynthesis of opioids in yeast. Science 349, 1095-1100 (2015).
Gerlt, J. A. et al. Enzyme function initiative-enzyme similarity tool (EFI-EST): A web tool for generating protein sequence similarity networks. Biochimica Et. Biophysica Acta (BBA)-Proteins Proteom. 1854, 1019-1037 (2015).
Gibson, D. G. et al. Enzymatic assembly of DNA molecules up to several hundred kilobases. Nature methods 6, 343-345 (2009).
Gietz, R. D. & Schiestl, R. H. Frozen competent yeast cells that can be transformed with high efficiency using the LiAc/SS carrier DNA/PEG method. Nat. Protoc. 2, 1 (2007).
Goetz, K. E., Coyle, C. M., Cheng, J. Z., O'Connor, S. E. & Panaccione, D. G. Ergot cluster-encoded catalase is required for synthesis of chanoclavine-I in Aspergillus fumigatus. Curr. Genet. 57, 201 (2011).
Haarmann, T., Ortel, I., Tudzynski, P. & Keller, U. Identification of the cytochrome P450 monooxygenase that bridges the clavine and ergoline alkaloid pathways. ChemBioChem 7, 645-652 (2006).
Hendrickson, J. B. & Wang, J. A new synthesis of lysergic acid. Org. Lett. 6, 3-5 (2004).
Inoue, H., Nojima, H. & Okayama, H. High efficiency transformation of Escherichia coli with plasmids. Gene 96, 23-28 (1990).
Jakubczyk, D. et al. Discovery and reconstitution of the cycloclavine biosynthetic pathway—enzymatic formation of a cyclopropyl group. Angew. Chem. Int. Ed. 54, 5117-5121 (2015).
Kozikowski, A. P. et al. Probing ergot alkaloid biosynthesis: Intermediates in the formation of ring C. J. Am. Chem. Soc. 115, 2482-2488 (1993).
Lieberman, A., Kupersmith, M., Estey, E. & Goldstein, M. Treatment of Parkinson's disease with bromocriptine. N. Engl. J. Med. 295, 1400-1404 (1976).
Liu, H. & Jia, Y. Ergot alkaloids: synthetic approaches to lysergic acid and clavine alkaloids. Nat. Prod. Rep. 34, 411-432 (2017).
Lorenz, N., Olšovská, J., Šulc, M. & Tudzynski, P. Alkaloid cluster gene ccsA of the ergot fungus Claviceps purpurea encodes chanoclavine I synthase, a flavin adenine dinucleotide-containing oxidoreductase mediating the transformation of Nmethyl-dimethylallyltryptophan to chanoclavine I. Appl. Environ. Microbiol. 76, 1822-1830 (2010).
Mantegani, S., Brambilla, E. & Varasi, M. Ergoline derivatives: receptor affinity and selectivity. II Farm. 54, 288-296 (1999).
Metzger, U. et al. The structure of dimethylallyl tryptophan synthase reveals a common architecture of aromatic prenyltransferases in fungi and bacteria. Proc. Natl Acad. Sci. 106, 14309-14314 (2009).
Nielsen, C. A. et al. The important ergot alkaloid intermediate chanoclavine-I produced in the yeast Saccharomyces cerevisiae by the combined action of EasC and EasE from Aspergillus japonicus. Microb. cell factories 13, 1 (2014).
Pertz, H. & Eich, E. Ergot alkaloids and their derivatives as ligands for serotoninergic, dopaminergic, and adrenergic receptors. Ergot: The Genus Claviceps. Amsterdam: Harwood Academic Publishers, 411-440 (1999).
Rigbers, O. & Li, S.-M. Ergot alkaloid biosynthesis in Aspergillus fumigatus overproduction and biochemical characterization of a 4-dimethylallyltryptophan N-methyltransferase. J. Biol. Chem. 283, 26859-26868 (2008).
Sanchez, R. G., Hahn-Hägerdal, B. & Gorwa-Grauslund, M. F. PGM2 overexpression improves anaerobic galactose fermentation in Saccharomyces cerevisiae. Microb. Cell Factories 9, 1-8 (2010).
Shannon, P. et al. Cytoscape: a software environment for integrated models of biomolecular interaction networks. Genome research 13, 2498-2504 (2003).
Tan, Y. Q. et al. Structure of a Minimal-Carboxysome-Derived Shell and Its Utility in Enzyme Stabilization. Biomacromolecules (2021).
Tandowsky, R. M. Clinical Evaluation of Combined Hydrogenated Ergot Alkaloids (Hydergine) in Arterial Hypertension: With Special Reference to Their Action in Central Manifestations. Circulation 9, 48-56 (1954).
Treco, D. A. & Lundblad, V. Preparation of Yeast Media. Curr. Protoc. Mol. Biol. 23, 13.11.11-13.11.17 (1993).
Umezaki, S., Yokoshima, S. & Fukuyama, T. Total synthesis of lysergic acid. Org. Lett. 15, 4230-4233 (2013).
Wacker, D. et al. Crystal structure of an LSD-bound human serotonin receptor. Cell 168, 377-389. e312 (2017).
Wallwey, C., Matuschek, M. & Li, S.-M. Ergot alkaloid biosynthesis in Aspergillus fumigatus: conversion of chanoclavine-I to chanoclavine-I aldehyde catalyzed by a short-chain alcohol dehydrogenase FgaDH. Arch. Microbiol. 192, 127-134 (2010).
Winblad, B. et al. Therapeutic use of nicergoline. Clin. drug Investig. 28, 533-552 (2008).
Zallot, R., Oberg, N. O. & Gerlt, J. A. ‘Democratized’genomic enzymology web tools for functional assignment. Current opinion in chemical biology 47, 77-85 (2018).
Zallot, R., Oberg, N. & Gerlt, J. A. The EFI Web Resource for Genomic Enzymology Tools: Leveraging Protein, Genome, and Metagenome Databases to Discover Novel Enzymes and Metabolic Pathways. Biochemistry 58, 4169-4182 (2019).

1. An isolated recombinant cell comprising one or more genes selected from the group consisting of dmaW, easF, easC, easE, easD, easAᵢₛₒₘₑᵣₐₛₑ, and cloA, wherein each gene codes for an enzyme from the biosynthetic pathway from tryptophan to D-lysergic acid (DLA).

2-3. (canceled)

4. The isolated recombinant cell according to claim 1, comprising at least one easE, at least one easAᵢₛₒₘₑᵣₐₛₑ and at least one cloA.

5. The isolated recombinant cell according to claim 4, wherein the easE comprises a sequence at least 80%, at least 85%, at least 90%, at least 95% identity to a sequence selected from the group consisting of SEQ ID NO: 6 to SEQ ID NO: 14, the easAᵢₛₒₘₑᵣₐₛₑ comprises a sequence at least 80%, at least 85%, at least 90%, at least 95% identity to a sequence selected from the group consisting of SEQ ID NO: 15 to SEQ ID NO: 18, and the cloA comprises a sequence at least 80%, at least 85%, at least 90%, at least 95% identity to SEQ ID NO: 22, 23, 24, 27 or 28.

6. The isolated recombinant cell according claim 4, wherein the easE comprises easE from Epichloe coenophiala (easE_Ec), easE from Aspergillus japonicas (easE_Aj) and/or east from Aspergillus indologenus, the easAᵢₛₒₘₑᵣₐₛₑ comprises easAᵢₛₒₘₑᵣₐₛₑ from Neotyphodium lolii (easA_Nl), easAᵢₛₒₘₑᵣₐₛₑ from Periglandula ipomoeae (easA_Pi), easAᵢₛₒₘₑᵣₐₛₑ from Claviceps purpurea (easA_Cp) and/or easAᵢₛₒₘₑᵣₐₛₑ from Epichloe coenophiala (easA_Ec), and the cloA comprises cloA from Claviceps paspali (CloA_Cpas), cloA from N. lolii (CloA_Nlol), cloA from P. ipomoeae (CloA_Pipo), cloA from E. coenophilia (CloA_XN6) and/or cloA from C. purpurea (CloA_Cpur).

7. The isolated recombinant cell according to claim 6, wherein the east from Epichloe coenophiala (easE_Ec) comprises a sequence at least 80%, at least 85%, at least 90%, at least 95% identity to SEQ ID NO: 14 and the east from Aspergillus japonicas (easE_Aj) or the easE from Aspergillus indologenus comprises a sequence at least 80%, at least 85%, at least 90%, at least 95% identity to SEQ ID NO: 6, the easAᵢₛₒₘₑᵣₐₛₑ from Neotyphodium lolii (easA_Nl) comprises a sequence at least 80%, at least 85%, at least 90%, at least 95% identity to SEQ ID NO: 15, the easAᵢₛₒₘₑᵣₐₛₑ from Claviceps purpurea (easA_Cp) comprises a sequence at least 80%, at least 85%, at least 90%, at least 95% identity to SEQ ID NO: 18, and the easAᵢₛₒₘₑᵣₐₛₑ from Epichloe coenophiala (easA_Ec) comprises a sequence at least 80%, at least 85%, at least 90%, at least 95% identity to SEQ ID NO: 17, and the cloA from E. coenophilia (CloA_XN6) comprises a sequence at least 80%, at least 85%, at least 90%, at least 95% identity to SEQ ID NO: 27 or 28 and the cloA from C. purpurea (CloA_Cpur) comprises a sequence at least 80%, at least 85%, at least 90%, at least 95% identity to SEQ ID NO: 22, 23 or 24.

8. The isolated recombinant cell according to claim 4, wherein the east comprises easE from Aspergillus japonicas (easE_Aj) or easE from Aspergillus indologenus, the easAᵢₛₒₘₑᵣₐₛₑ comprises easAᵢₛₒₘₑᵣₐₛₑ from Epichloe coenophialia (easA_Ec) and the cloA comprises cloA from C. purpurea (CloA_Cpur).

9. The isolated recombinant cell according to claim 4, wherein the east comprises a sequence at least 80%, at least 85%, at least 90%, at least 95% identity to SEQ ID NO: 6, the easAᵢₛₒₘₑᵣₐₛₑ comprises a sequence at least 80%, at least 85%, at least 90%, at least 95% identity to SEQ ID NO: 17 and the cloA comprises a sequence at least 80%, at least 85%, at least 90%, at least 95% identity to SEQ ID NO: 23.

10. The isolated recombinant cell according to claim 4, wherein the east expresses an enzyme, which catalyses conversion of 4-Dimethylallyl-L-abrine to Chanoclavine I, the easAᵢₛₒₘₑᵣₐₛₑ expresses an enzyme which catalyses conversion of Chanoclavine-I aldehyde to agroclavine, and the cloA expresses an enzyme which catalyses conversion of agroclavine to D-lysergic acid.

11-31. (canceled)

32. The isolated recombinant cell according to claim 4, wherein the recombinant yeast cell further comprises at least one dmaW, at least one easF, and at least one easD.

33. The isolated recombinant cell according to claim 32, wherein the dmaW comprises a sequence at least 80%, at least 85%, at least 90%, at least 95% identity to SEQ ID NO: 1, the easF comprises a sequence at least 80%, at least 85%, at least 90%, at least 95% identity to SEQ ID NO: 2; and the easD comprises a sequence at least 80%, at least 85%, at least 90%, at least 95% identity to SEQ ID NO: 4.

34. The isolated recombinant cell according to claim 32, wherein the dmaW, easF and easD each expresses a respective enzyme, wherein each respective enzyme is functional.

35. (canceled)

36. The isolated recombinant cell according to claim 32, wherein the recombinant yeast cell further comprises at least one easC and/or at least one easG.

37. The isolated recombinant cell according to claim 36, wherein the easC comprises a sequence at least 80%, at least 85%, at least 90%, at least 95% identity to SEQ ID NO: 3 and the easG comprises a sequence at least 80%, at least 85%, at least 90%, at least 95% identity to SEQ ID NO: 5.

38. An isolated recombinant cell comprising at least one dmaW, at least one easF, at least one easE or easC, at least one easD, at least one easAᵢₛₒₘₑᵣₐₛₑ or easG, and at least one cloA gene.

39. An isolated recombinant cell comprising at least one dmaW, at least one easF, at least one easE, at least one easD, at least one easAᵢₛₒₘₑᵣₐₛₑ, and at least one cloA gene.

40. An isolated recombinant cell comprising at least one dmaW, at least one easF, at least one easC, at least one easD, at least one easAᵢₛₒₘₑᵣₐₛₑ, at least one easG, and at least one cloA gene.

41. The isolated recombinant cell according to claim 40, wherein the dmaW comprises a sequence at least 80%, at least 85%, at least 90%, at least 95% identity to SEQ ID NO: 1; easF comprises a sequence at least 80%, at least 85%, at least 90%, at least 95% identity to SEQ ID NO: 2; easE comprises a sequence at least 80%, at least 85%, at least 90%, at least 95% identity to SEQ ID NO: 6; easC comprises a sequence at least 80%, at least 85%, at least 90%, at least 95% identity to SEQ ID NO: 3; easD comprises a sequence at least 80%, at least 85%, at least 90%, at least 95% identity to SEQ ID NO: 4; easAᵢₛₒₘₑᵣₐₛₑ comprises a sequence at least 80%, at least 85%, at least 90%, at least 95% identity to SEQ ID NO: 17; easG comprises a sequence at least 80%, at least 85%, at least 90%, at least 95% identity to SEQ ID NO: 5; and cloA comprises a sequence at least 80%, at least 85%, at least 90%, at least 95% identity to SEQ ID NO: 23.

42. An isolated recombinant cell according to claim 32, further comprising multiple copies of FAD1 and PDI1 genes, wherein the FAD1 comprises a sequence at least 80%, at least 85%, at least 90%, at least 95% identity to SEQ ID NO: 35 and PDI1 comprises a sequence at least 80%, at least 85%, at least 90%, at least 95% identity to SEQ ID NO: 36.

43. (canceled)

44. An isolated recombinant cell according to claim 42, capable of expressing the one or more genes, wherein expression of the one or more genes produces respective gene product(s), wherein the respective gene product(s) are respective enzyme(s).

45-49. (canceled)

50. A method of culturing the recombinant cell according to claim 1 in an appropriate culture medium, for preparing D-lysergic acid.

51. (canceled)
